(19) United States
(12) Patent Application Publication
(10) Pub. No.: US 2025/0151774 A1
(43) Pub. Date: May 15, 2025

(54) NICOTINE SALT FORMULATIONS FOR AEROSOL DEVICES AND METHODS THEREOF

(71) Applicant: JUUL Labs, Inc., San Francisco, CA (US)

(72) Inventors: Adam Bowen, San Francisco, CA (US); Chenyue Xing, San Francisco, CA (US)

(21) Appl. No.: 19/024,843

(22) Filed: Jan. 16, 2025

Related U.S. Application Data
(63) Continuation of application No. 18/939,841, filed on Nov. 7, 2024.
Application No. 18/939,841 is a continuation of application No. 17/171,976, filed on Feb. 9, 2021, now Pat. No. 12,156,533.
Application No. 17/171,976 is a continuation of application No. 14/925,961, filed on Oct. 28, 2015, now Pat. No. 10,952,468.
Application No. 14/925,961 is a continuation of application No. 14/271,071, filed on May 6, 2014, now abandoned.
(60) Provisional application No. 61/912,507, filed on Dec. 5, 2013.
(60) Provisional application No. 61/820,128, filed on May 6, 2013.

(51) Int. Cl. A24B 15/167; A24B 15/16; A24F 40/10; A61K 9/00; A61K 9/12; A61K 31/465; A61K 31/60; A61K 47/10

(52) U.S. Cl. CPC A24B 15/167; A24B 15/16; A61K 9/007; A61K 9/12; A61K 31/465; A61K 31/60; A61K 47/10; A24F 40/10

(57) ABSTRACT

A nicotine salt liquid formulation for generating an inhalable aerosol in an electronic cigarette comprising nicotine salt that forms about 0.5% to about 20% nicotine is provided.

### CROSS-REFERENCE TO RELATED APPLICATIONS

This application is a continuation of U.S. application Ser. No. 18/939,841 filed Nov. 7, 2024, which is a continuation of U.S. application Ser. No. 17/171,976 filed Feb. 9, 2021, issued as U.S. Pat. No. 12,156,533, which is a continuation of U.S. application Ser. No. 14/925,961 filed Oct. 28, 2015, issued as U.S. Pat. No. 10,952,468, which is a continuation of U.S. application Ser. No. 14/271,071 filed May 6, 2014, abandoned, which claims the benefit of priority to U.S. Application No. 61/912,507 filed Dec. 5, 2013, and U.S. Application No. 61/820,128 filed May 6, 2013, which are incorporated herein by reference in their entireties. This application is related to U.S. application Ser. No. 14/512,311, issued as U.S. Pat. No. 9,215,895, and to U.S. application Ser. No. 14/925,968, abandoned.

### SUMMARY OF THE INVENTION

Provided herein is a method of delivering nicotine to a user comprising operating an electronic cigarette to a user wherein the electronic cigarette comprises a nicotine salt formulation comprising a nicotine salt in a biologically acceptable liquid carrier wherein an acid used to form said nicotine salt is characterized by vapor pressure >20 mmHg at 200° C., and inhaling an aerosol generated from the nicotine salt formulation heated by the electronic cigarette.

Provided herein is a method of delivering nicotine to a user comprising operating an electronic cigarette to a user wherein the electronic cigarette comprises a nicotine salt formulation comprising a nicotine salt in a biologically acceptable liquid carrier wherein an acid used to form said nicotine salt is characterized by vapor pressure of about 20 to 200 mmHg at 200° C., and inhaling an aerosol generated from the nicotine salt formulation heated by the electronic cigarette.

Provided herein is a method of delivering nicotine to a user comprising operating an electronic cigarette wherein the electronic cigarette comprises a nicotine salt formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point, and inhaling an aerosol generated from the nicotine salt formulation heated by the electronic cigarette.

Provided herein is a method of delivering nicotine to a user comprising providing an electronic cigarette to a user wherein the electronic cigarette comprises a nicotine salt formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point, and inhaling an aerosol generated from the nicotine salt formulation heated by the electronic cigarette.

Provided herein is a method of delivering nicotine to the blood of a user, said method comprising providing an aerosol that is inhaled by the user from an electronic cigarette that comprises a nicotine salt formulation wherein providing the aerosol comprises the electronic cigarette heating the formulation thereby generating the aerosol, wherein the aerosol is effective in delivering a level of nicotine in the blood of the user that is at least 5 ng/ml at about 1.5 minutes after a first puff of ten puffs of the aerosol, each puff taken at 30 second intervals.

Provided herein is a nicotine salt liquid formulation in an electronic cigarette for generating an inhalable aerosol upon heating in the electronic cigarette, the formulation in the cigarette comprising a nicotine salt in a biologically acceptable liquid carrier wherein an acid used to form said nicotine salt is characterized by vapor pressure >20 mmHg at 200° C.

Provided herein is a nicotine salt liquid formulation in an electronic cigarette for generating an inhalable aerosol upon heating in the electronic cigarette, the formulation in the cigarette comprising a nicotine salt in a biologically acceptable liquid carrier wherein an acid used to form said nicotine salt is characterized by vapor pressure of about 20 to 200 mmHg at 200° C.

Provided herein is a nicotine salt liquid formulation in an electronic cigarette for generating an inhalable aerosol upon heating in the electronic cigarette, the formulation in the cigarette comprising a nicotine salt in a biologically acceptable liquid carrier wherein an acid used to form said nicotine salt is further characterized by a melting point <160° C., a boiling point >160° C., and at least a 50-degree difference between the melting point and the boiling point.

Provided herein is a nicotine salt liquid formulation in an electronic cigarette for generating an inhalable aerosol upon heating in the electronic cigarette, the formulation in the cigarette comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Provided herein is a nicotine salt liquid formulation for generating an inhalable aerosol upon heating in the electronic cigarette, the nicotine salt liquid formulation comprising a nicotine salt in a biologically acceptable liquid carrier wherein an acid used to form said nicotine salt is characterized by vapor pressure >20 mmHg at 200° C.

Provided herein is a nicotine salt liquid formulation for generating an inhalable aerosol upon heating in the electronic cigarette, the nicotine salt liquid formulation comprising a nicotine salt in a biologically acceptable liquid carrier wherein an acid used to form said nicotine salt is characterized by vapor pressure of about 20 to 200 mmHg at 200° C.

Provided herein is a nicotine salt liquid formulation for generating an inhalable aerosol upon heating in the electronic cigarette, the nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point.

Provided herein is a nicotine salt liquid formulation for generating an inhalable aerosol upon heating in the electronic cigarette, the nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Provided herein is a nicotine salt liquid formulation for use in an electronic cigarette the nicotine salt liquid formulation comprising a nicotine salt in a biologically acceptable liquid carrier wherein an acid used to form said nicotine salt is characterized by vapor pressure >20 mmHg at 200° C.

Provided herein is a nicotine salt liquid formulation for use in an electronic cigarette the nicotine salt liquid formulation comprising a nicotine salt in a biologically acceptable liquid carrier wherein an acid used to form said nicotine salt is characterized by vapor pressure of about 20 to 200 mmHg at 200° C.

Provided herein is a nicotine salt liquid formulation for use in an electronic cigarette the nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point.

Provided herein is a nicotine salt liquid formulation for use in an electronic cigarette the nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Provided herein is a use of a nicotine salt formulation for delivery of nicotine to a user from an electronic cigarette wherein the nicotine salt formulation comprises a nicotine salt in a biologically acceptable liquid carrier wherein an acid used to form said nicotine salt is characterized by vapor pressure >20 mmHg at 200° C., and the nicotine salt formulation is heated by the electronic cigarette to generate an aerosol inhalable by the user.

Provided herein is a use of a nicotine salt formulation for delivery of nicotine to a user from an electronic cigarette wherein the nicotine salt formulation comprises a nicotine salt in a biologically acceptable liquid carrier wherein an acid used to form said nicotine salt is characterized by vapor pressure of about 20 to 200 mmHg at 200° C., and the nicotine salt formulation is heated by the electronic cigarette to generate an aerosol inhalable by the user.

Provided herein is a use of a nicotine salt formulation for delivery of nicotine to a user from an electronic cigarette wherein the nicotine salt formulation comprises a nicotine salt in a biologically acceptable liquid carrier wherein an acid used to form said nicotine salt is further characterized by a melting point <160° C., a boiling point >160° C., and at least a 50-degree difference between the melting point and the boiling point, and the nicotine salt formulation is heated by the electronic cigarette to generate an aerosol inhalable by the user.

Provided herein is a use of a nicotine salt formulation for delivery of nicotine to the blood of a user from an electronic cigarette, wherein the nicotine salt formulation in the electronic cigarette is heated to form an aerosol which delivers a level of nicotine in the blood of the user that is at least 5 ng/ml at about 1.5 minutes after a first puff of ten puffs of the aerosol, each puff taken at 30 second intervals.

Provided herein is a use of a nicotine salt formulation for delivery of nicotine to a user from an electronic cigarette wherein the nicotine salt formulation comprises a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point, and the nicotine salt formulation is heated by the electronic cigarette to generate an aerosol inhalable by the user.

Provided herein is a cartomizer for an electronic cigarette comprising: a nicotine salt liquid formulation comprising a nicotine salt in a biologically acceptable liquid carrier wherein an acid used to form said nicotine salt is characterized by vapor pressure >20 mmHg at 200° C.; an atomizer comprising a heating element in fluid communication with the nicotine salt liquid formulation; and a fluid storage compartment that stores the nicotine salt liquid formulation.

Provided herein is a cartomizer for an electronic cigarette comprising: a nicotine salt liquid formulation comprising a nicotine salt in a biologically acceptable liquid carrier wherein an acid used to form said nicotine salt is characterized by vapor pressure of about 20 to 200 mmHg at 200° C.; an atomizer comprising a heating element in fluid communication with the nicotine salt liquid formulation; and a fluid storage compartment that stores the nicotine salt liquid formulation.

Provided herein is a cartomizer for an electronic cigarette comprising: a nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point; an atomizer comprising a heating element in fluid communication with the nicotine salt liquid formulation; and a fluid storage compartment that stores the nicotine salt liquid formulation.

Provided herein is a cartomizer for an electronic cigarette comprising: a nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point; an atomizer comprising a heating element in fluid communication with the nicotine salt liquid formulation; and a fluid storage compartment that stores the nicotine salt liquid formulation.

Provided herein is an electronic cigarette for generating an inhalable aerosol comprising: a fluid storage compartment; a heater; and a nicotine salt liquid formulation in the fluid storage compartment, the liquid formulation comprising a nicotine salt in a biologically acceptable liquid carrier wherein an acid used to form said nicotine salt is characterized by vapor pressure >20 mmHg at 200° C.; a battery; and a mouthpiece.

Provided herein is an electronic cigarette for generating an inhalable aerosol comprising: a fluid storage compartment; a heater; and a nicotine salt liquid formulation in the fluid storage compartment, the liquid formulation comprising a nicotine salt in a biologically acceptable liquid carrier wherein an acid used to form said nicotine salt is characterized by vapor pressure of about 20 to 200 mmHg at 200° C.; a battery; and a mouthpiece.

Provided herein is an electronic cigarette for generating an inhalable aerosol comprising: a fluid storage compartment; a heater; and a nicotine salt liquid formulation in the fluid storage compartment, the liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point; a battery; and a mouthpiece.

Provided herein is an electronic cigarette for generating an inhalable aerosol comprising: a fluid storage compartment; a heater; and a nicotine salt liquid formulation in the fluid storage compartment, the liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point; a battery; and a mouthpiece.

Provided herein is a cartridge in an electronic cigarette comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure >20 mmHg at 200° C.

Provided herein is a cartridge in an electronic cigarette comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure of about 20 to 200 mmHg at 200° C.

Provided herein is a cartridge in an electronic cigarette comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point.

Provided herein is a cartridge in an electronic cigarette comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Provided herein is a kit comprising: (a) an electronic cigarette for generating an inhalable aerosol comprising (i) a device body comprising a cartridge receptacle; (ii) a cartridge comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure >20 mmHg at 200° C.; (iii) a heater; (iv) a battery; and (v) a mouthpiece; and (b) instructions for using the electronic cigarette to generate an inhalable aerosol.

Provided herein is a kit comprising: (a) an electronic cigarette for generating an inhalable aerosol comprising (i) a device body comprising a cartridge receptacle; (ii) a cartridge comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure of about 20 to 200 mmHg at 200° C.; (iii) a heater; (iv) a battery; and (v) a mouthpiece; and (b) instructions for using the electronic cigarette to generate an inhalable aerosol.

Provided herein is a kit comprising: (a) an electronic cigarette for generating an inhalable aerosol comprising (i) a device body comprising a cartridge receptacle; (ii) a cartridge comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point; (iii) a heater; (iv) a battery; and (v) a mouthpiece; and (b) instructions for using the electronic cigarette to generate an inhalable aerosol.

Provided herein is a kit comprising: (a) an electronic cigarette for generating an inhalable aerosol comprising (i) a device body comprising a cartridge receptacle; (ii) a cartridge comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point; (iii) a heater; (iv) a battery; and (v) a mouthpiece; and (b) instructions for using the electronic cigarette to generate an inhalable aerosol.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are used, and the accompanying drawings of which:

FIG. 1 illustrates results of heart rate data measured for six minutes from start of puffing. Y-axis is heart rate (bpm) and X-axis represent duration of the test (−60 to 180 seconds);

FIG. 2 illustrates results of heart rate data measured for ten minutes from start of puffing. Y-axis is heart rate (bpm) and X-axis represents duration of the test (0 to 10 minutes);

FIG. 3 illustrates the calculated vapor pressures of various acids relative to nicotine;

FIG. 4 illustrates the pharmacokinetic profiles for eight test articles in a blood plasma study;

FIG. 5 illustrates the comparison of Cₘₐₓ and Tₘₐₓ for eight test articles in a blood plasma study;

FIG. 6 illustrates the comparison of Cₘₐₓ and AUC for eight test articles in a blood plasma study;

FIG. 7 depicts an example embodiment of an electronic cigarette having a fluid storage compartment comprising an embodiment nicotine salt formulation described herein; and

FIG. 8 depicts an example embodiment of an electronic cigarette cartomizer having a fluid storage compartment, a heater, and comprising an embodiment nicotine salt formulation described herein.

### DETAILED DESCRIPTION OF THE INVENTION

Nicotine is a chemical stimulant and increases heart rate and blood pressure when provided to an individual or animal. Nicotine transfer to an individual is associated with a feeling of physical and/or emotional satisfaction. Conflicting reports have been published regarding the transfer efficiency of free base nicotine in comparison to mono- or di-protonated nicotine salts. Studies on the transfer efficiency of free base nicotine and nicotine salts are complex and have yielded unpredictable results. Further, such transfer efficiency studies have been performed under extremely high temperature conditions, comparable to smoking; therefore, they offer scant guidance on the transfer efficiency of free base nicotine and nicotine salts under low-temperature vaporization conditions. Some reports have posited that nicotine free base should give rise to a greater satisfaction in a user than any corresponding nicotine salt.

It has been unexpectedly discovered herein that certain nicotine salt formulations provide satisfaction in an individual superior to that of free base nicotine, and more comparable to the satisfaction in an individual smoking a traditional cigarette. The satisfaction effect is consistent with an efficient transfer of nicotine to the lungs of an individual and a rapid rise of nicotine absorption in the plasma as shown, for non-limiting example, in Example 8, at least. It has also been unexpectedly discovered herein that certain nicotine salt formulations provide greater satisfaction than other nicotine salt formulations, and such effect has been shown in blood plasma levels of example nicotine salt formulations herein, for non-limiting example, in Example 8, at least. These results show a difference in rate of nicotine uptake in the blood that is higher for some nicotine salt formulations aerosolized by an electronic cigarette than for other nicotine salt formulations, and likewise higher than nicotine freebase formulations, while the peak concentration of the nictoine in the blood and total amount of nicotine delivered appears comparable to a traditional cigarette, and do not appear to vary significantly between the various nicotine formulations. Therefore, described herein are nicotine salt formulations for use in an electronic cigarette, or the like, that provide a general satisfaction effect consistent with an efficient transfer of nicotine to the lungs of an individual and a rapid rise of nicotine absorption in the plasma. Provided herein, therefore, are devices, formulation of nicotine salts, systems, cartomizers, kits and methods that are used to inhale an aerosol generated from a nicotine salt liquid formulation through the mouth or nose as described herein or as would be obvious to one of skill in the art upon reading the disclosure herein.

Consistent with these satisfaction effects, it has unexpectedly been found herein that there is a difference between the Cₘₐₓ (maximum concentration) and Tₘₐₓ (time at which the maximum concentration is measured) when measuring blood plasma nicotine levels of freebase nicotine formulations inhaled using a low temperature vaporization device, i.e. electronic cigarette, as compared to the Cₘₐₓ and Tₘₐₓ (similarly measuring blood plasma nicotine levels) of a traditional cigarette. Also consistent with these satisfaction effects, it has unexpectedly been found herein that there is a difference between the Cₘₐₓ (maximum concentration) and Tₘₐₓ (time at which the maximum concentration is measured) when measuring blood plasma nicotine levels of freebase nicotine formulations inhaled using a low temperature vaporization device, i.e. electronic cigarette, as compared to the Cₘₐₓ and Tₘₐₓ (similarly measuring blood plasma nicotine levels) of nicotine salt formulations inhaled using a low temperature vaporization device, i.e. electronic cigarette. Additionally, it has unexpectedly been found that there is a difference between the rate of nicotine uptake in the plasma of users inhaling freebase nicotine formulations using a low temperature vaporization device, i.e. electronic cigarette, as compared to the rate of nicotine uptake in the plasma of users inhaling smoke of a traditional cigarette. Furthermore, it has unexpectedly been found that there is a difference between the rate of nicotine uptake in the plasma of users inhaling freebase nicotine formulations using a low temperature vaporization device, i.e. electronic cigarette, as compared to the rate of nicotine uptake in the plasma of users inhaling nicotine salt formulations using a low temperature vaporization device, i.e. electronic cigarette.

Thus, looking at freebase nicotine as a source of nicotine in compositions used in e-cigarettes, freebase nicotine compositions' delivery of nicotine to blood when inhaled using is not necessarily comparable in blood plasma levels (Cₘₐₓ and Tₘₐₓ) to a traditional cigarette's nicotine delivery to blood when inhaled. Freebase nicotine compositions' delivery of nicotine to blood when inhaled using is not necessarily comparable in blood plasma levels (Cₘₐₓ and Tₘₐₓ) to nicotine salt formulations' nicotine delivery to blood when inhaled. Freebase nicotine compositions' delivery of nicotine to blood when inhaled using is not necessarily comparable in blood plasma levels when measuring the rate of nicotine uptake in the blood within the first 0-5 minutes to a traditional cigarette's nicotine delivery to blood when inhaled. Freebase nicotine compositions' delivery of nicotine to blood when inhaled using necessarily is not comparable in blood plasma levels when measuring the rate of nicotine uptake in the blood within the first 0-5 minutes to nicotine salt formulations' nicotine delivery to blood when inhaled.

Also consistent with these satisfaction effects, it has unexpectedly been found herein that while there appears to be comparable Cₘₐₓ and Tₘₐₓ values (measuring blood plasma nicotine levels) of nicotine salt formulations inhaled using a low temperature vaporization device, i.e. electronic cigarette, as compared to the Cₘₐₓ and Tₘₐₓ (similarly measuring blood plasma nicotine levels) of a traditional cigarette, there is a demonstrable difference between the rate of nicotine uptake in the plasma of users inhaling certain nicotine salt formulations using a low temperature vaporization device, i.e. electronic cigarette, as compared to the rate of nicotine uptake in the plasma of users inhaling other nicotine salt formulations using a low temperature vaporization device, i.e. electronic cigarette. It is also unexpected that while the Cₘₐₓ and Tₘₐₓ values are comparable to those of a traditional cigarette, (or are approaching that of a traditional cigarette), the rate of nicotine uptake in the plasma of blood of users is higher in certain nicotine salt formulations than that of the traditional cigarette. The nicotine salt formulations which demonstrate the quickest rate of nicotine uptake in the plasma were more preferred in satisfaction evaluations, and were rated more equivalent to cigarette satisfaction than the nicotine salt formulations showing the slowest rates of rise of nicotine in the subjects' blood plasma. In addition, doubling the concentration of the nicotine salt in the formulation may not necessarily impact the rate of absorption of nicotine in the blood (see, for non-limiting example Example 8, nicotine benzoate tested in 4% and 2% concentrations).

Thus, looking at nicotine salt formulations used in e-cigarettes, nicotine salt formulations delivered using an e-cigarette appear comparable in Cₘₐₓ and Tₘₐₓ values (measuring blood plasma nicotine levels), however, not all nicotine salts perform similarly to each other or to a traditional cigarette with respect to the rate of nicotine uptake in the blood at early time periods (0-1.5 minutes). These results are unexpected. Nicotine salt formulations made using acids having a Vapor Pressure between 20-300 mmHg @ 200° C., or Vapor Pressure >20 mmHg @ 200° C., or a Vapor Pressure from 20 to 300 mmHg @ 200° C., or a Vapor Pressure from 20 to 200 mmHg @ 200° C., a Vapor Pressure between 20 and 300 mmHg @ 200° C. appear to have a higher rate of nicotine uptake in the blood at early time periods (0-1.5 minutes, 0-3 minutes, 0-2 minutes, 0-4 minutes for non-limiting example) than other nicotine salt formulations, however, they also provide satisfaction comparable to a traditional cigarette or closer to a traditional cigarette (as compared to other nicotine salt formulations or as compared to nicotine freebase formulations). For non-limiting example, acids that meet one or more criteria of the prior sentence include salicylic acid, sorbic acid, benzoic acid, lauric acid, and levulinic acid. Nicotine salt formulations made using acids that have a difference between boiling point and melting point of at least 50° C., and a boiling point greater than 160° C., and a melting point less than 160° C. appear to have a higher rate of nicotine uptake in the blood at early time periods (0-1.5 minutes, 0-3 minutes, 0-2 minutes, 0-4 minutes for non-limiting example) than other nicotine salt formulations, however, they also provide satisfaction comparable to a traditional cigarette or closer to a traditional cigarette (as compared to other nicotine salt formulations or as compared to nicotine freebase formulations). For non-limiting example, acids that meet the criteria of the prior sentence include salicylic acid, sorbic acid, benzoic acid, pyruvic acid, lauric acid, and levulinic acid. Nicotine salt formulations made using acids that have a difference between boiling point and melting point of at least 50° C., and a boiling point at most 40° C. less than operating temperature, and a melting point at least 40° C. lower than operating temperature appear to have a higher rate of nicotine uptake in the blood at early time periods (0-1.5 minutes, 0-3 minutes, 0-2 minutes, 0-4 minutes for non-limiting example) than other nicotine salt formulations, however, they also provide satisfaction comparable to a traditional cigarette or closer to a traditional cigarette (as compared to other nicotine salt formulations or as compared to nicotine freebase formulations). Operating temperature can be 100° C. to 300° C., or about 200° C., about 150° C. to about 250° C., 180 C to 220° C., about 180° C. to about 220° C., 185° C. to 215° C., about 185° C. to about 215° C., about 190° C. to about 210° C., 190° C. to 210° C., 195° C. to 205° C., or about 195° C. to about 205° C. For non-limiting example, acids that meet the criteria of the prior sentence include salicylic acid, sorbic acid, benzoic acid, pyruvic acid, lauric acid, and levulinic acid. Combinations of these criteria for preference of certain nicotine salt formulations are contemplated herein.

Other reasons for excluding certain acids from formulations may be unrelated to the rate of nicotine uptake, however. For example, an acid may be inappropriate for use with the device materials (corrosive or otherwise incompatible). Sulfuric acid is an example of this, which may be inappropriate for the e-cigarette device. An acid may be inappropriate for use in inhalation or for toxicity reasons—thus not be compatible for human consumption, ingestion, or inhalation. Sulfuric acid again is an example of this, which may be inappropriate for a user of an e-cigarette device, depending on the embodiment of the composition. An acid that is bitter or otherwise bad-tasting may also provide a reason for exclusion, such as acetic acid in some embodiments. Acids that oxidize at room temperature or at operating temperature may be inappropriate for certain embodiments, for example, sorbic acid, as this indicates a decomposition or reaction or instability that may be undesirable in the formulation. Decomposition of acids at room or operating temperatures may also indicate that the acid is inappropriate for use in the embodiment formulations. For example, citric acid decomposes at 175° C., and malic acid decomposes at 140° C., thus for a device operating at 200° C., these acids may not be appropriate. Acids that have poor solubility in the composition constituents may be inappropriate for use in certain embodiments of the compositions herein. For example, nicotine bitartrate with a composition of nicotine and tartaric acid as 1:2 molar ratio will not produce a solution at a concentration of 0.5% (w/w) nicotine or higher and 0.9% (w/w) tartaric acid or higher in propylene glycol (PG) or vegetable glycerin (VG) or any mixture of PG and VG at ambient conditions. As used herein, weight percentage (w/w) refers to the weight of the individual component over the weight of the total formulation.

As used in this specification and the claims, the singular forms “a,” “an,” and “the” include plural referents unless the context clearly dictates otherwise.

The term “organic acid” as used herein, refers to an organic compound with acidic properties (e.g., by Brønsted-Lowry definition, or Lewis definition). A common organic acid is the carboxylic acids, whose acidity is associated with their carboxyl group-COOH. A dicarboxylic acid possesses two carboxylic acid groups. The relative acidity of an organic is measured by its pKₐ value and one of skill in the art knows how to determine the acidity of an organic acid based on its given pKa value. The term “keto acid” as used herein, refers to organic compounds that contain a carboxylic acid group and a ketone group. Common types of keto acids include alpha-keto acids, or 2-oxoacids, such as pyruvic acid or oxaloacetic acid, having the keto group adjacent to the carboxylic acid; beta-keto acids, or 3-oxoacids, such as acetoacetic acid, having the ketone group at the second carbon from the carboxylic acid; gamma-keto acids, or 4-oxoacids, such as levulinic acid, having the ketone group at the third carbon from the carboxylic acid.

The term “electronic cigarette” or “e-cigarette” or “low temperature vaporization device” as used herein, refers to an electronic inhaler that vaporizes a liquid solution into an aerosol mist, simulating the act of tobacco smoking. The liquid solution comprises a formulation comprising nicotine. There are many electronic cigarettes which do not resemble conventional cigarettes at all. The amount of nicotine contained can be chosen by the user via the inhalation. In general, an electronic cigarette contains three essential components: a plastic cartridge that serves as a mouthpiece and a reservoir for liquid, an “atomizer” that vaporizes the liquid, and a battery. Other embodiment electronic cigarettes include a combined atomizer and reservoir, called a “cartomizer” that may or may not be disposable, a mouthpiece that may be integrated with the cartomizer or not, and a battery.

As used in this specification and the claims, unless otherwise stated, the term “about” refers to variations of 1%, 2%, 3%, 4%, 5%, 10%, 15%, or 25%, depending on the embodiment.

Suitable carriers (e.g., a liquid solvent) for the nicotine salts described herein include a medium in which a nicotine salt is soluble at ambient conditions, such that the nicotine salt does not form a solid precipitate. Examples include, but are not limited to, glycerol, propylene glycol, trimethylene glycol, water, ethanol and the like, as well as combinations thereof. In some embodiments, the liquid carrier comprises 0% to 100% of propylene glycol and 100% to 0% of vegetable glycerin. In some embodiments, the liquid carrier comprises 10% to 70% of propylene glycol and 90% to 30% of vegetable glycerin. In some embodiments, the liquid carrier comprises 20% to 50% of propylene glycol and 80% to 50% of vegetable glycerin. In some embodiments, the liquid carrier comprises 30% propylene glycol and 70% vegetable glycerin.

The formulations described herein vary in concentration. In some formulations, a dilute concentration of the nicotine salt in the carrier is utilized. In some formulations, a less dilute concentration of the nicotine salt in the carrier is utilized. In some formulations the concentration of nicotine in the nicotine salt formulation is about 1% (w/w) to about 25% (w/w). In some formulations the concentration of nicotine in the nicotine salt formulation is about 1% (w/w) to about 20% (w/w). In some formulations the concentration of nicotine in the nicotine salt formulation is about 1% (w/w) to about 18% (w/w). In some embodiments the concentration of nicotine in the nicotine salt formulation is about 1% (w/w) to about 15% (w/w). In some formulations the concentration of nicotine in the nicotine salt formulation is about 4% (w/w) to about 12% (w/w). In some formulations the concentration of nicotine in the nicotine salt formulation is about 4% (w/w). In some embodiments the concentration of nicotine in the nicotine salt formulation is about 2% (w/w). In some formulations the concentration of nicotine in the nicotine salt formulation is 1% (w/w) to 25% (w/w). In some formulations the concentration of nicotine in the nicotine salt formulation is 1% (w/w) to 20% (w/w). In some formulations the concentration of nicotine in the nicotine salt formulation is 1% (w/w) to 18% (w/w). In some formulations the concentration of nicotine in the nicotine salt formulation is 1% (w/w) to 15% (w/w). In some formulations the concentration of nicotine in the nicotine salt formulation is 4% (w/w) to 12% (w/w). In some formulations the concentration of nicotine in the nicotine salt formulation is 4% (w/w). In some formulations the concentration of nicotine in the nicotine salt formulation is 2% (w/w). In some formulations, a less dilute concentration of one nicotine salt is used in conjunction with a more dilute concentration of a second nicotine salt. In some formulations, the concentration of nicotine in the first nicotine salt formulation is about 1% to about 20%, and is combined with a second nicotine salt formulation having a concentration of nicotine therein from about 1% to about 20% or any range or concentration therein. In some formulations, the concentration of nicotine in the first nicotine salt formulation is 1% to 20%, and is combined with a second nicotine salt formulation having a concentration of nicotine therein from 1% to 20% or any range or concentration therein. As used with respect to concentrations of nicotine in the nicotine salt formulations, the term “about” refers to ranges of 0.05% (i.e. if the concentration is about 2%, the range is 1.95%-2.05%), 0.1 (i.e. if the concentration is about 2%, the range is 1.9%-2.1%), 0.25 (i.e. if the concentration is about 2%, the range is 1.75%-2.25%), 0.5 (i.e. if the concentration is about 2%, the range is 1.5%-2.5%), or 1 (i.e. if the concentration is about 4%, the range is 3%-5%), depending on the embodiment.

Nicotine salts are formed by the addition of a suitable acid, including organic or inorganic acids. In some formulations provided herein, suitable organic acids are carboxylic acids. Examples of organic carboxylic acids disclosed herein are monocarboxylic acids, dicarboxylic acids (organic acid containing two carboxylic acid groups), carboxylic acids containing an aromatic group such as benzoic acids, hydroxycarboxylic acids, heterocyclic carboxylic acids, terpenoid acids, sugar acids; such as the pectic acids, amino acids, cycloaliphatic acids, aliphatic carboxylic acids, keto carboxylic acids, and the like. In some formulations provided herein, the organic acids used herein are monocarboxylic acids. Nicotine salts are formed from the addition of a suitable acid to nicotine. In some formulations provided herein, the stoichiometric ratios of the nicotine to acid (nicotine:acid) are 1:1, 1:2, 1:3, 1:4, 2:3, 2:5, 2:7, 3:4, 3:5, 3:7, 3:8, 3:10, 3:11, 4:5, 4:7, 4:9, 4:10, 4:11, 4:13, 4:14, 4:15, 5:6, 5:7, 5:8, 5:9, 5:11, 5:12, 5:13, 5:14, 5:16, 5:17, 5:18, or 5:19. In some formulations provided herein, the stoichiometric ratios of the nicotine to acid are 1:1, 1:2, 1:3, or 1:4 (nicotine:acid).

Nicotine is an alkaloid molecule that comprises two basic nitrogens. It may occur in different states of protonation. For example, if no protonation exists, nicotine is referred to as the “free base.” If one nitrogen is protonated, then the nicotine would be “mono-protonated.”

Nicotine salt formulations may be formed by adding a suitable acid to nicotine, stirring the neat mixture at ambient temperature or at elevated temperature, and then diluting the neat mixture with a carrier mixture, such as a mixture of propylene glycol and glycerin. In some embodiments, the suitable acid is completely dissolved by the nicotine prior to dilution. The suitable acid may not completely dissolved by the nicotine prior to dilution. The addition of the suitable acid to the nicotine to form a neat mixture may cause an exothermic reaction. The addition of the suitable acid to the nicotine to form a neat mixture may be conducted at 55° C. The addition of the suitable acid to the nicotine to form a neat mixture may be conducted at 90° C. The neat mixture may be cooled to ambient temperature prior to dilution. The dilution may be carried out at elevated temperature.

Nicotine salt formulations may be prepared by combining nicotine and a suitable acid in a carrier mixture, such as a mixture of propylene glycol and glycerin. The mixture of nicotine and a first carrier mixture is combined with a mixture of a suitable acid in a second carrier mixture. In some embodiments, the first and second carrier mixtures are identical in composition. In some embodiments, the first and second carrier mixtures are not identical in composition. In some embodiments, heating of nicotine/acid/carrier mixture is required to facilitate complete dissolution.

In some embodiments, nicotine salt formulations may be prepared and added to a solution of 3:7 ratio by weight of propylene glycol (PG)/vegetable glycerin (VG), and mixed thoroughly. While described herein as producing 10 g of each of the formulations, all procedures noted infra are scalable. Other manners of formulation may also be employed form the formulations noted infra, without departing from the disclosure herein, and as would be known to one of skill in the art upon reading the disclosure herein.

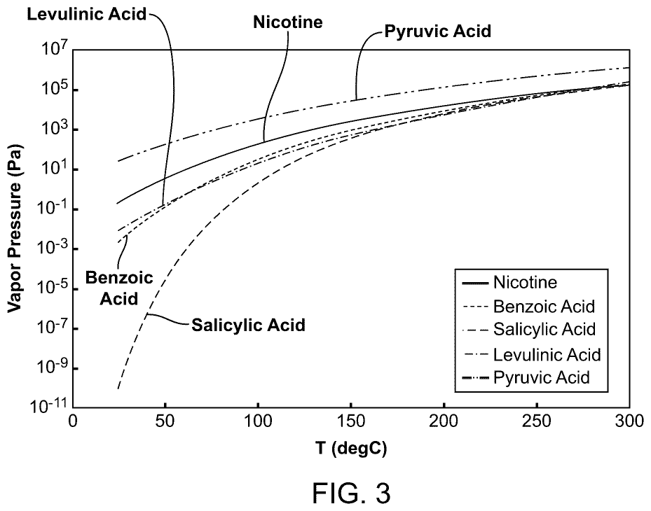

The optimal nicotine salt formulation may be determined by the vapor pressure of the constituent acid. In some embodiments, the nicotine salt formulations comprise an acid with a vapor pressure that is similar to the vapor pressure of free base nicotine. In some embodiments, the nicotine salt formulations are formed from an acid with a vapor pressure that is similar to the vapor pressure of free base nicotine at the heating temperature of the device. FIG. 3 illustrates this trend. Nicotine salts formed from nicotine and benzoic acid; nicotine and salicylic acid; or nicotine and levulinic acid are salts that produce a satisfaction in an individual user consistent with efficient transfer of nicotine and a rapid rise in nicotine plasma levels. This pattern may be due to the mechanism of action during heating of the nicotine salt formulation. The nicotine salt may disassociate at, or just below, the heating temperature of the device, resulting in a mixture of free base nicotine and the individual acid. At that point, if both the nicotine and acid have similar vapor pressures, they may aerosolize at the same time, giving rise to a transfer of both free base nicotine and the constituent acid to the user.

The nicotine salt liquid formulation for generating an inhalable aerosol upon heating in an electronic cigarette may comprise a nicotine salt in a biologically acceptable liquid carrier; wherein the acid used to form said nicotine salt is characterized by a vapor pressure between 20-4000 mmHg at 200° C. In some embodiments, the acid used to form the nicotine salt is characterized by vapor pressure between 20-2000 mmHg at 200° C. In some embodiments, the acid used to form the nicotine salt is characterized by vapor pressure between 100-300 mmHg at 200° C.

Unexpectedly, different nicotine salt formulations produced varying degrees of satisfaction in an individual. In some embodiments, the extent of protonation of the nicotine salt affected satisfaction, such that more protonation was less satisfying as compared to less protonation. The nicotine salt formed may be monoprotonated. The nicotine salt formed may be diprotonated. he nicotine salt may exist in more than one protonation state, e.g., an equilibrium of mono-protonated and di-protonated nicotine salts. The extent of protonation of the nicotine molecule may be dependent upon the stoichiometric ratio of nicotine:acid used in the salt formation reaction. The extent of protonation of the nicotine molecule may be dependent upon the solvent. The extent of protonation of the nicotine molecule may be unknown. In some embodiments, monoprotonated nicotine salts produced a high degree of satisfaction in the user. For example, nicotine benzoate and nicotine salicylate are mono-protonated nicotine salts and all produce a high degree of satisfaction in the user. The reason for this trend may be explained by a mechanism of action wherein the nicotine is first deprotonated prior to transfer to the vapor with the constituent acid and then retained and stabilized after re-protonated by the acid going down stream to the lungs of the user. It may be easier to remove one proton versus two protons, thus resulting in better transfer efficiency. In addition, the lack of satisfaction of free base nicotine indicates that a second factor may be important. A nicotine salt may be best performing when it is at its optimal extent of protonation, depending on the salt. For example, nicotine pyruvate is a nicotine salt with 1:2 nicotine:acid ratio. The formulation containing nicotine pyruvate (1:2) may deliver more satisfaction to the user than the one containing same amount of nicotine but only half amount of pyruvic acid, i.e. nicotine pyruvate (1:1). This may be explained as 1 mole of nicotine produces a salt with 2 moles of pyruvic acid. When there is not enough pyruvic acid to associate with all nicotine molecules, the free base nicotine left unprotonated in the formulation may reduce the satisfaction the formulation provides.

The flavor of the constituent acid used in the salt formation may be a consideration in choosing the acid. A suitable acid may have minimal or no toxicity to humans in the concentrations used. A suitable acid may be compatible with the electronic cigarette components it contacts or could contact at the concentrations used. That is, such acid does not degrade or otherwise react with the electronic cigarette components it contacts or could contact. The odor of the constituent acid used in the salt formation may be a consideration in choosing a suitable acid. The concentration of the nicotine salt in the carrier may affect the satisfaction in the individual user. In some embodiments, the flavor of the formulation is adjusted by changing the acid. In some embodiments, the flavor of the formulation is adjusted by adding exogenous flavorants. In some embodiments, an unpleasant tasting or smelling acid is used in minimal quantities to mitigate such characteristics. In some embodiments, exogenous pleasant smelling or tasting acid is added to the formulation. Examples of salts which can provide flavor and aroma to the mainstream aerosol at certain levels include nicotine acetate, nicotine oxalate, nicotine malate, nicotine isovalerate, nicotine lactate, nicotine citrate, nicotine phenylacetate and nicotine myristate.

Nicotine salt formulations may generate an inhalable aerosol upon heating in an electronic cigarette. The amount of nicotine or nicotine salt aerosol inhaled may be user-determined. The user may, for example, modify the amount of nicotine or nicotine salt inhaled by adjusting his inhalation strength.

Formulations are described herein comprising two or more nicotine salts. In some embodiments, wherein a formulation comprises two or more nicotine salts, each individual nicotine salt is formed as described herein.

Nicotine salt formulations, as used herein, refer to a single or mixture of nicotine salts with other suitable chemical components used for e-cigarette, such as carriers, stabilizers, diluents, dispersing agents, suspending agents, thickening agents, and/or excipients. In certain embodiments, the nicotine salt formulation is stirred at ambient conditions for 20 minutes. In certain embodiments, the nicotine salt formulation is heated and stirred at 55 C for 20 minutes. In certain embodiments, the nicotine salt formulation is heated and stirred at 90 C for 60 minutes. In certain embodiments, the formulation facilitates administration of nicotine to an organism (e.g., lung).

The nicotine of nicotine salt formulations provided herein is either naturally occurring nicotine (e.g., from extract of nicotineous species such as tobacco), or synthetic nicotine. In some embodiments, the nicotine is (−)-nicotine, (+)-nicotine, or a mixture thereof. In some embodiments, the nicotine is employed in relatively pure form (e.g., greater than about 80% pure, 85% pure, 90% pure, 95% pure, or 99% pure). In some embodiments, the nicotine for nicotine salt formulation provided herein is “water clear” in appearance in order to avoid or minimize the formation of tarry residues during the subsequent salt formation steps.

Nicotine salt formulations used for e-cigarettes described herein, in some embodiments, have a nicotine concentration of about 0.5% (w/w) to about 20% (w/w), wherein the concentration is of nicotine weight to total solution weight, i.e. (w/w). In certain embodiments, nicotine salt formulations provided herein have a nicotine concentration of about 1% (w/w) to about 20% (w/w). In certain embodiments, nicotine salt formulations provided herein have a nicotine concentration of about 1% (w/w) to about 18% (w/w). In certain embodiments, nicotine salt formulations provided herein have a nicotine concentration of about 1% (w/w) to about 15% (w/w). In certain embodiments, nicotine salt formulations provided herein have a nicotine concentration of about 4% (w/w) to about 12% (w/w). In certain embodiments, nicotine salt formulations provided herein have a nicotine concentration of about 1% (w/w) to about 18% (w/w), about 3% (w/w) to about 15% (w/w), or about 4% (w/w) to about 12% (w/w). In certain embodiments, nicotine salt formulations provided herein have a nicotine concentration of about 0.5% (w/w) to about 10% (w/w). In certain embodiments, nicotine salt formulations provided herein have a nicotine concentration of about 0.5% (w/w) to about 5% (w/w). In certain embodiments, nicotine salt formulations provided herein have a nicotine concentration of about 0.5% (w/w) to about 4% (w/w). In certain embodiments, nicotine salt formulations provided herein have a nicotine concentration of about 0.5% (w/w) to about 3% (w/w). In certain embodiments, nicotine salt formulations provided herein have a nicotine concentration of about 0.5% (w/w) to about 2% (w/w). In certain embodiments, nicotine salt formulations provided herein have a nicotine concentration of about 0.5% (w/w) to about 1% (w/w). In certain embodiments, nicotine salt formulations provided herein have a nicotine concentration of about 1% (w/w) to about 10% (w/w). In certain embodiments, nicotine salt formulations provided herein have a nicotine concentration of about 1% (w/w) to about 5% (w/w). In certain embodiments, nicotine salt formulations provided herein have a nicotine concentration of about 1% (w/w) to about 4% (w/w). In certain embodiments, nicotine salt formulations provided herein have a nicotine concentration of about 1% (w/w) to about 3% (w/w). In certain embodiments, nicotine salt formulations provided herein have a nicotine concentration of about 1% (w/w) to about 2% (w/w). In certain embodiments, nicotine salt formulations provided herein have a nicotine concentration of about 2% (w/w) to about 10% (w/w). In certain embodiments, nicotine salt formulations provided herein have a nicotine concentration of about 2% (w/w) to about 5% (w/w). In certain embodiments, nicotine salt formulations provided herein have a nicotine concentration of about 2% (w/w) to about 4% (w/w). Certain embodiments provide a nicotine salt formulation having a nicotine concentration of about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.5%, 5.0%, 5.5%, 6.0%, 6.5%, 7.0%, 7.5%, 8.0%, 8.5%, 9.0%, 9.5%, 10%, 11%, 12%, 13%, 14%, 15%, 16%, 17%, 18%, 19%, or 20% (w/w), or more, including any increments therein. Certain embodiments provide a nicotine salt formulation having a nicotine concentration of about 5% (w/w). Certain embodiments provide a nicotine salt formulation having a nicotine concentration of about 4% (w/w). Certain embodiments provide a nicotine salt formulation having a nicotine concentration of about 3% (w/w). Certain embodiments provide a nicotine salt formulation having a nicotine concentration of about 2% (w/w). Certain embodiments provide a nicotine salt formulation having a nicotine concentration of about 1% (w/w). Certain embodiments provide a nicotine salt formulation having a nicotine concentration of about 0.5% (w/w).

The formulation further may comprise one or more flavorants.

Thee suitable acid for the nicotine salt formulation may have a vapor pressure >20 mmHg at 200° C. and is non-corrosive to the electronic cigarette or is non-toxic to humans. In some embodiments, the suitable acid for nicotine salt formation is selected from the group consisting of salicylic acid, formic acid, sorbic acid, acetic acid, benzoic acid, pyruvic acid, lauric acid, and levulinic acid.

Thee suitable acid for the nicotine salt formulation may have a vapor pressure of about 20 to 200 mmHg at 200° C. and is non-corrosive to the electronic cigarette or is non-toxic to humans. In some embodiments, the suitable acid for nicotine salt formation is selected from the group consisting of salicylic acid, benzoic acid, lauric acid, and levulinic acid.

Thee suitable acid for the nicotine salt formulation may have a melting point <160° C., a boiling point >160° C., at least a 50-degree difference between the melting point and the boiling point, and is non-corrosive to the electronic cigarette or is non-toxic to humans. In some embodiments, the suitable acid for nicotine salt formation has a melting point at least 40 degrees lower than the operating temperature of the electronic cigarette, a boiling point no more than 40 degrees lower than the operating temperature of the electronic cigarette, at least a 50-degree difference between the melting point and the boiling point, and is non-corrosive to the electronic cigarette or is non-toxic to humans; wherein the operating temperature is 200° C. In some embodiments, the suitable acid for nicotine salt formation is selected from the group consisting of salicylic acid, sorbic acid, benzoic acid, pyruvic acid, lauric acid, and levulinic acid.

Thee suitable acid for the nicotine salt formulation does not decompose at the operating temperature of the electronic cigarette. In some embodiments, the suitable acid for nicotine salt formation does not oxidize at the operating temperature of the electronic cigarette. In some embodiments, the suitable acid for nicotine salt formation does not oxidize at room temperature. In some embodiments, the suitable acid for nicotine salt formation does not provide an unpleasant taste. In some embodiments, the suitable acid for nicotine salt formation has good solubility in a liquid formulation for use in an electronic cigarette.

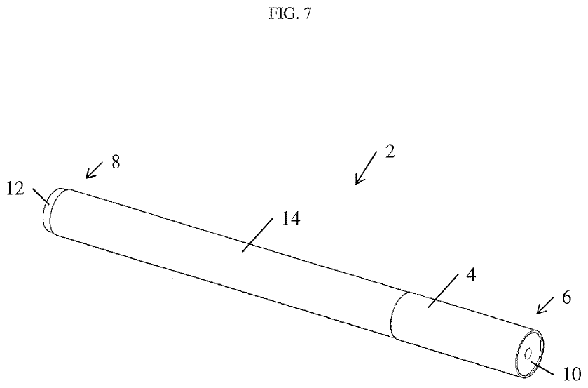

Provided herein is an electronic cigarette 2 having a fluid storage compartment 4 comprising an embodiment nicotine salt formulation of any embodiment described herein within the fluid storage compartment described herein. An embodiment is shown in FIG. 7. The electronic cigarette 2 of FIG. 7 includes a mouth end 6, and a charging end 8. The mouth-end 6 includes a mouthpiece 10. The charging end 8 may connect to a battery or a charger or both, wherein the battery is within a body of the electronic cigarette, and the charger is separate from the battery and couples to the body or the battery to charge the battery. In some embodiments the electronic cigarette comprises a rechargeable battery within a body 14 of the electronic cigarette and the charge end 8 comprises a connection 12 for charging the rechargeable battery. In some embodiments, the electronic cigarette comprises a cartomizer that comprises the fluid storage compartment and an atomizer. In some embodiments, the atomizer comprises a heater. In some embodiments the fluid storage compartment 4 is separable from an atomizer. In some embodiments the fluid storage compartment 4 is replaceable as part of a replaceable cartridge. In some embodiments the fluid storage compartment 4 is refillable. In some embodiments, the mouthpiece 10 is replaceable.

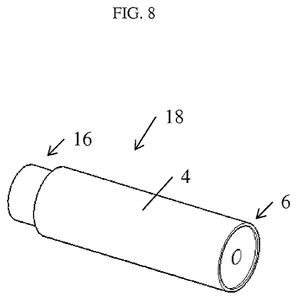

Provided herein is a cartomizer 18 for an electronic cigarette 2 having a fluid storage compartment 4 comprising an embodiment nicotine salt formulation of any embodiment described herein within the fluid storage compartment described herein. The cartomizer 18 embodiment of FIG. 8 includes a mouth end 6, and a connection end 16. The connection end 16 in the embodiment of FIG. 8 couples the cartomizer 14 to a body of an electronic cigarette, or to a battery of the electronic cigarette, or both. The mouth end 6 includes a mouthpiece 10. In some embodiments, the cartomizer does not include a mouthpiece, and in such embodiments, the cartomizer can be coupled to a mouthpiece of an electronic cigarette, or the cartomizer can be coupled to a battery or body of an electronic cigarette, while the mouthpiece is also coupled to the battery or the body of the electronic cigarette. In some embodiments, the mouthpiece is integral with the body of the electronic cigarette. In some embodiments, including the embodiment of FIG. 8, the cartomizer 18 comprises the fluid storage compartment 4 and an atomizer (not shown). In some embodiments, the atomizer comprises a heater (not shown)

### EXAMPLES

### Example 1

### Preparation of Nicotine Salt Formulations

Various nicotine formulations were prepared and added to a solution of 3:7 ratio by weight of propylene glycol (PG)/vegetable glycerin (VG), and mixed thoroughly. The examples shown below were used to make 10 g of each of the formulations. All procedures are scalable.

For example, in order to make nicotine formulations with a final nicotine free base equivalent concentration of 2% (w/w), the following procedures were applied to each individual formulation.

Nicotine benzoate salt formulation: 0.15 g benzoic acid was added to a beaker followed by adding 0.2 g nicotine to the same beaker. The mixture was stirred at 55° C. for 20 minutes until benzoic acid was completely dissolved and an orange oily mixture was formed. The mixture was cooled down to ambient conditions. 9.65 g PG/VG (3:7) solution was added to the orange nicotine benzoate salt and the mixture was stirred until a visually homogenous formulation solution was achieved.)

Nicotine benzoate salt formulation can also be made by adding 0.15 g benzoic acid to a beaker followed by adding 0.2 g nicotine and 9.65 g PG/VG (3:7) solution to the same beaker. The mixture was then stirred at 55° C. for 20 minutes until a visually homogenous formulation solution was achieved with no undissolved chemicals.

Nicotine citrate salt formulation was made by adding 0.47 g citric acid to a beaker followed by adding 0.2 g nicotine and 9.33 g PG/VG (3:7) solution to the same beaker. The mixture was then stirred at 90° C. for 60 minutes until a visually homogenous formulation solution was achieved with no undissolved chemicals.

Nicotine malate salt formulation was made by adding 0.33 g L-malic acid to a beaker followed by adding 0.2 g nicotine and 9.47 g PG/VG (3:7) solution to the same beaker. The mixture was then stirred at 90° C. for 60 minutes until a visually homogenous formulation solution was achieved with no undissolved chemicals.

Nicotine succinate salt formulation was made by adding 0.29 g succinic acid to a beaker followed by adding 0.2 g nicotine and 9.51 g PG/VG (3:7) solution to the same beaker. The mixture was then stirred at 90° C. for 60 minutes until a visually homogenous formulation solution was achieved with no undissolved chemicals.

Nicotine salicylate salt formulation was made by adding 0.17 g salicylic acid to a beaker followed by adding 0.2 g nicotine and 9.63 g PG/VG (3:7) solution to the same beaker. The mixture was then stirred at 90° C. for 60 minutes until a visually homogenous formulation solution was achieved with no undissolved chemicals.

Nicotine salicylate salt formulation can also be made by adding 0.17 g salicylic acid to a beaker followed by adding 0.2 g nicotine to the same beaker. The mixture was stirred at 90° C. for 60 minutes until salicylic acid was completely dissolved and an orange oily mixture was formed. The mixture was either cooled to ambient conditions or kept at 90° C. when 9.63 g PG/VG (3:7) solution was added. The mixture was then stirred at 90° C. until a visually homogenous formulation solution was achieved with no undissolved chemicals.

Nicotine free base formulation was made by adding 0.2 g nicotine to a beaker followed by adding 9.8 g PG/VG (3:7) solution to the same beaker. The mixture was then stirred at ambient conditions for 10 minutes until a visually homogenous formulation solution was achieved.

For example, in order to make nicotine salt formulations with a final nicotine free base equivalent concentration of 3% (w/w), the following procedures were applied to each individual formulation.

Nicotine benzoate salt formulation: 0.23 g benzoic acid was added to a beaker followed by adding 0.3 g nicotine to the same beaker. The mixture was stirred at 55° C. for 20 minutes until benzoic acid was completely dissolved and an orange oily mixture was formed. The mixture was cooled down to ambient conditions. 9.47 g PG/VG (3:7) solution was added to the orange nicotine benzoate salt and the blend was stirred until a visually homogenous formulation solution was achieved.

Nicotine benzoate salt formulation can also be made by adding 0.23 g benzoic acid to a beaker followed by adding 0.3 g nicotine and 9.47 g PG/VG (3:7) solution to the same beaker. The mixture was then stirred at 55° C. for 20 minutes until a visually homogenous formulation solution was achieved with no undissolved chemicals.

Nicotine citrate salt formulation was made by adding 0.71 g citric acid to a beaker followed by adding 0.3 g nicotine and 8.99 g PG/VG (3:7) solution to the same beaker. The mixture was then stirred at 90° C. for 60 minutes until a visually homogenous formulation solution was achieved with no undissolved chemicals.

Nicotine malate salt formulation was made by adding 0.5 g L-malic acid to a beaker followed by adding 0.3 g nicotine and 9.2 g PG/VG (3:7) solution to the same beaker. The mixture was then stirred at 90° C. for 60 minutes until a visually homogenous formulation solution was achieved with no undissolved chemicals.

Nicotine levulinate salt formulation was made by adding melted 0.64 g levulinic acid to a beaker followed by adding 0.3 g nicotine to the same beaker. The mixture was stirred at ambient conditions for 10 minutes. Exothermic reaction took place and oily product was produced. The mixture was allowed to cool down to ambient temperature and 9.06 g PG/VG (3:7) solution was added to the same beaker. The mixture was then stirred at ambient conditions for 20 minutes until a visually homogenous formulation solution was achieved.

Nicotine pyruvate salt formulation was made by adding 0.33 g pyruvic acid to a beaker followed by adding 0.3 g nicotine to the same beaker. The mixture was stirred at ambient conditions for 10 minutes. Exothermic reaction took place and oily product was produced. The mixture was allowed to cool down to ambient temperature and 9.37 g PG/VG (3:7) solution was added to the same beaker. The mixture was then stirred at ambient conditions for 20 minutes until a visually homogenous formulation solution was achieved.

Nicotine succinate salt formulation was made by adding 0.44 g succinic acid to a beaker followed by adding 0.3 g nicotine and 9.26 g PG/VG (3:7) solution to the same beaker. The mixture was then stirred at 90° C. for 60 minutes until a visually homogenous formulation solution was achieved with no undissolved chemicals.

Nicotine salicylate salt formulation was made by adding 0.26 g salicylic acid to a beaker followed by adding 0.3 g nicotine and 9.44 g PG/VG (3:7) solution to the same beaker. The mixture was then stirred at 90° C. for 60 minutes until a visually homogenous formulation solution was achieved with no undissolved chemicals.

Nicotine salicylate salt formulation can also be made by adding 0.26 g salicylic acid to a beaker followed by adding 0.3 g nicotine to the same beaker. The mixture was stirred at 90° C. for 60 minutes until salicylic acid was completely dissolved and an orange oily mixture was formed. The mixture was either cooled to ambient conditions or kept at 90° C. when 9.44 g PG/VG (3:7) solution was added. The blend was then stirred at 90 C until a visually homogenous formulation solution was achieved with no undissolved chemicals.

Nicotine free base formulation was made by adding 0.3 g nicotine to a beaker followed by adding 9.7 g PG/VG (3:7) solution to the same beaker. The mixture was then stirred at ambient conditions for 10 minutes until a visually homogenous formulation solution was achieved.

For example, in order to make nicotine salt formulations with a final nicotine free base equivalent concentration of 4% (w/w), the following procedures were applied to each individual formulation.

Nicotine benzoate salt formulation: 0.3 g benzoic acid was added to a beaker followed by adding 0.4 g nicotine to the same beaker. The mixture was stirred at 55° C. for 20 minutes until benzoic acid was completely dissolved and an orange oily mixture was formed. The mixture was cooled down to ambient conditions. 9.7 g PG/VG (3:7) solution was added to the orange nicotine benzoate salt and the blend was stirred until a visually homogenous formulation solution was achieved.

Nicotine benzoate salt formulation can also be made by adding 0.3 g benzoic acid to a beaker followed by adding 0.4 g nicotine and 9.7 g PG/VG (3:7) solution to the same beaker. The mixture was then stirred at 55° C. for 20 minutes until a visually homogenous formulation solution was achieved with no undissolved chemicals.

For example, in order to make nicotine salt formulations with a final nicotine free base equivalent concentration of 5% (w/w), the following procedures were applied to each individual formulation.

Nicotine benzoate salt formulation: 0.38 g benzoic acid was added to a beaker followed by adding 0.5 g nicotine to the same beaker. The mixture was stirred at 55° C. for 20 minutes until benzoic acid was completely dissolved and an orange oily mixture was formed. The mixture was cooled down to ambient conditions. 9.12 g PG/VG (3:7) solution was added to the orange nicotine benzoate salt and the blend was stirred until a visually homogenous formulation solution was achieved.

Nicotine benzoate salt formulation can also be made by adding 0.38 g benzoic acid to a beaker followed by adding 0.5 g nicotine and 9.12 g PG/VG (3:7) solution to the same beaker. The mixture was then stirred at 55° C. for 20 minutes until a visually homogenous formulation solution was achieved with no undissolved chemicals.

Nicotine malate salt formulation was made by adding 0.83 g L-malic acid to a beaker followed by adding 0.5 g nicotine and 8.67 g PG/VG (3:7) solution to the same beaker. The mixture was then stirred at 90° C. for 60 minutes until a visually homogenous formulation solution was achieved with no undissolved chemicals.

Nicotine levulinate salt formulation was made by adding melted 1.07 g levulinic acid to a beaker followed by adding 0.5 g nicotine to the same beaker. The mixture was stirred at ambient conditions for 10 minutes. Exothermic reaction took place and oily product was produced. The mixture was allowed to cool down to ambient temperature and 8.43 g PG/VG (3:7) solution was added to the same beaker. The mixture was then stirred at ambient conditions for 20 minutes until a visually homogenous formulation solution was achieved.

Nicotine pyruvate salt formulation was made by adding 0.54 g pyruvic acid to a beaker followed by adding 0.5 g nicotine to the same beaker. The mixture was stirred at ambient conditions for 10 minutes. Exothermic reaction took place and oily product was produced. The mixture was allowed to cool down to ambient temperature and 8.96 g PG/VG (3:7) solution was added to the same beaker. The mixture was then stirred at ambient conditions for 20 minutes until a visually homogenous formulation solution was achieved.

Nicotine succinate salt formulation was made by adding 0.73 g succinic acid to a beaker followed by adding 0.5 g nicotine and 8.77 g PG/VG (3:7) solution to the same beaker. The mixture was then stirred at 90° C. for 60 minutes until a visually homogenous formulation solution was achieved with no undissolved chemicals.

Nicotine salicylate salt formulation was made by adding 0.43 g salicylic acid to a beaker followed by adding 0.5 g nicotine and 9.07 g PG/VG (3:7) solution to the same beaker. The mixture was then stirred at 90° C. for 60 minutes until a visually homogenous formulation solution was achieved with no undissolved chemicals.

Nicotine salicylate salt formulation can also be made by adding 0.43 g salicylic acid to a beaker followed by adding 0.5 g nicotine to the same beaker. The mixture was stirred at 90° C. for 60 minutes until salicylic acid was completely dissolved and an orange oily mixture was formed. The mixture was either cooled to ambient conditions or kept at 90 C when 9.07 g PG/VG (3:7) solution was added. The blend was then stirred at 90° C. until a visually homogenous formulation solution was achieved with no undissolved chemicals.

Nicotine free base formulation was made by adding 0.5 g nicotine to a beaker followed by adding 9.5 g PG/VG (3:7) solution to the same beaker. The mixture was then stirred at ambient conditions for 10 minutes until a visually homogenous formulation solution was achieved.

Various formulations comprising different nicotine salts can be prepared similarly, or different concentrations of the above-noted nicotine formulations or other nicotine salt formulations can be prepared as one of skill in the art would know to do upon reading the disclosure herein.

Various formulations comprising two or more nicotine salts can be prepared similarly in a solution of 3:7 ratio of propylene glycol (PG)/vegetable glycerin (VG). For example, 0.43 g (2.5% w/w nicotine) of nicotine levulinate salt and 0.34 g (2.5% w/w nicotine) of nicotine acetate salt are added to 9.23 g of PG/VG solution, to achieve a 5% w/w nicotine formulation.

Also provided is another exemplary formulation. For example, 0.23 g (1.33% w/w nicotine) of nicotine benzoate salt (molar ratio 1:1 nicotine/benzoic acid), 0.25 g (1.33% w/w nicotine) of nicotine salicylate salt (molar ratio 1:1 nicotine/salicylic acid) and 0.28 g (1.34% w/w nicotine) of nicotine pyruvate salt (molar ratio 1:2 nicotine/pyruvic acid) are added to 9.25 g of PG/VG solution, to achieve a 5% w/w nicotine formulation.

### Example 2

Heart Rate Study of Nicotine Solutions Via e-Cigarette

Exemplary formulations of nicotine levulinate, nicotine benzoate, nicotine succinate, nicotine salicylate, nicotine malate, nicotine pyruvate, nicotine citrate, nicotine freebase, and a control of propylene glycol were prepared as noted in Example 1 in 3% w/w solutions and were administered in the same fashion by an electronic cigarette to the same human subject. About 0.5 mL of each solution was loaded into an “eRoll” cartridge atomizer (joyetech.com) to be used in the study. The atomizer was then attached to an “eRoll” e-cigarette (same manufacturer). The operating temperature was from about 150° C. to about 250° C., or from about 180° C. to about 220° C.

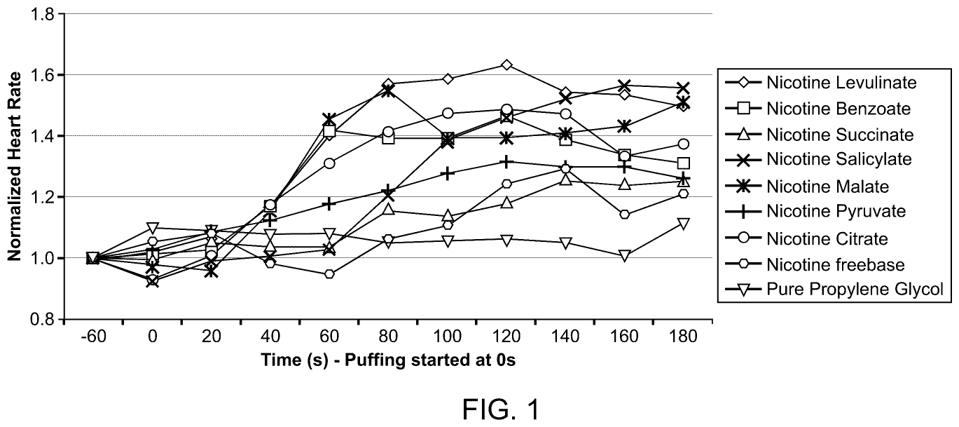

Heart rate measurements were taken for 6 minutes; from 1 minute before start of puffing, for 3 minutes during puffing, and continuing until 2 minutes after end of puffing. The test participant took 10 puffs over 3 minutes in each case. The base heart rate was the average heart rate over the first 1 minute before start of puffing. Heart rate after puffing started was averaged over 20-second intervals. Puffing (inhalation) occurred every 20 seconds for a total of 3 minutes. Normalized heart rate was defined as the ratio between individual heart rate data point and the base heart rate. Final results were presented as normalized heart rate, shown for the first 4 minutes in FIG. 1.

FIG. 1 summarizes results from heart rate measurements taken for a variety of nicotine salt formulations. For ease of reference in reviewing FIG. 1, at the 180-second timepoint, from top to bottom (highest normalized heart rate to lowest normalized heart rate), the nicotine formulations are as follows: nicotine salicylate formulation, nicotine malate formulation, nicotine levulinate formulation (nearly identical to nicotine malate formulation at 180 seconds, thus, as a second reference point: the nicotine malate formulation curve is lower than the nicotine levulinate formulation curve at the 160-second time point), nicotine pyruvate formulation, nicotine benzoate formulation, nicotine citrate formulation, nicotine succinate formulation, and nicotine free base formulation. The bottom curve (lowest normalized heart rate) at the 180-second timepoint is associated with the placebo (100% propylene glycol). The test formulations comprising a nicotine salt cause a faster and more significant rise in heart rate than the placebo. The test formulations comprising a nicotine salt also cause faster and more significant rise when compared with a nicotine freebase formulation with the same amount of nicotine by weight. In addition, the nicotine salts (e.g., nicotine benzoate and nicotine pyruvate) prepared from the acids having calculated vapor pressures between 20-200 mmHg at 200° C. (benzoic acid (171.66 mmHg), with the exception of pyruvic acid (having a boiling point of 165 C), respectively) cause a faster rise in heart rate than the rest. The nicotine salts (e.g., nicotine levulinate, nicotine benzoate, and nicotine salicylate) prepared from the acids (benzoic acid, levulinic acid and salicylic acid, respectively) also cause a more significant heart rate increase. Thus, other suitable nicotine salts formed by the acids with the similar vapor pressure and/or similar boiling point may be used in accordance with the practice of the present invention. This experience of increased heart rate theoretically approaching or theoretically comparable to that of a traditional burned cigarette has not been demonstrated or identified in other electronic cigarette devices. Nor has it been demonstrated or identified in low temperature tobacco vaporization devices (electronic cigarettes) that do not burn the tobacco, even when a nicotine salt was used (a solution of 20% (w/w) or more of nicotine salt) as an additive to the tobacco. Thus the results from this experiment are surprising and unexpected.

### Example 3

Satisfaction Study of Nicotine Salt Solution Via e-Cigarette

In addition to the heart rate study shown in Example 2, nicotine formulations (using 3% w/w nicotine formulations as described in Example 1) were used to conduct a satisfaction study in a single test participant. The test participant, an e-cigarette and/or traditional cigarette user, was required to have no nicotine intake for at least 12 hours before the test. The participant took 10 puffs using an e-cigarette (same as used in Example 2) over 3 minutes in each case, and then was asked to rate the level of physical and emotional satisfaction he or she felt on a scale of 0-10, with 0 being no physical or emotional satisfaction. The results indicated that the least satisfying compound was the nicotine free base. Nicotine benzoate, nicotine salicylate, and nicotine succinate all performed well, followed by nicotine pyruvate, nicotine citrate, and nicotine pyruvate.

Based on the Satisfaction Study, the nicotine salts formulations with acids having vapor pressure ranges between >20 mmHg @ 200° C., or 20-200 mmHg @ 200° C., or 100-300 mmHg @ 200° C. provide more satisfaction than the rest (except the pyruvic acid which has boiling point of 165° C.). For reference, it has been determined that salicylic acid has a vapor pressure of about 135.7 mmHg @ 200° C., benzoic acid has a vapor pressure of about 171.7 mmHg @ 200° C., lauric acid has a vapor pressure of about 38 mmHg @ 200° C., and levulinic acid has a vapor pressure of about 149 mmHg @ 200° C.

### Example 4

### Test Formulation 1 (TF1):

A solution of nicotine levulinate in glycerol comprising nicotine salt used: 1.26 g (12.6% w/w) of 1:3 nicotine levulinate 8.74 g (87.4% w/w) of glycerol-Total weight 10.0 g.

Neat nicotine levulinate was added to the glycerol, and mixed thoroughly. L-Nicotine has a molar mass of 162.2 g, and levulinic acid molar mass is 116.1 g. In a 1:3 molar ratio, the percentage of nicotine in nicotine levulinate by weight is given by: 162.2 g/(162.2 g+ (3×116.1 g))=31.8% (w/w).

### Example 5

### Test Formulation 2 (TF2):

A solution of free base nicotine in glycerol comprising 0.40 g (4.00% w/w) of L-nicotine was dissolved in 9.60 g (96.0% w/w) of glycerol and mixed thoroughly.

### Example 6

Heart Rate Study of Nicotine Solutions Via e-Cigarette:

Both formulations (TF1 and TF2) were administered in the same fashion by an electronic cigarette to the same human subject: about 0.6 mL of each solution was loaded into “eGo-C” cartridge atomizer (joyetech.com). The atomizer was then attached to an “eVic” e-cigarette (same manufacturer). This model of e-cigarette allows for adjustable voltage, and therefore wattage, through the atomizer. The operating temperature of the e-cigarette is from about 150° C. to about 250° C., or from about 180° C. to about 220° C.

The atomizer in both cases has resistance 2.4 ohms, and the e-cigarette was set to 4.24V, resulting in 7.49 W of power. (P=V\hat{ }2/R)

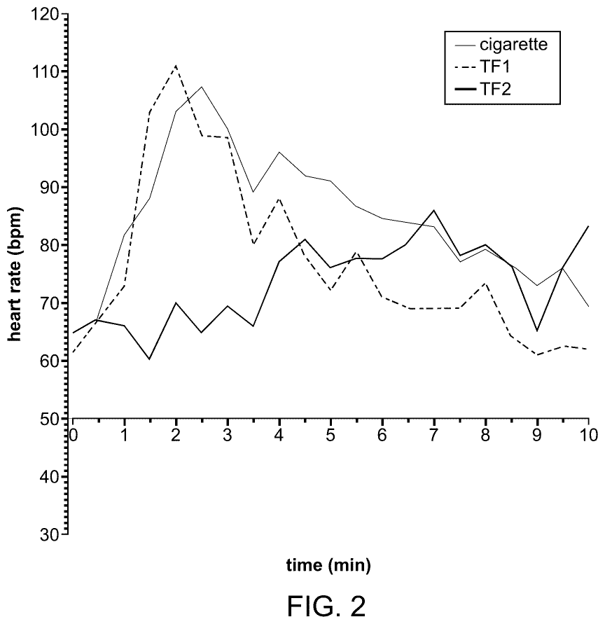

Heart rate was measured in a 30-second interval for ten minutes from start of puffing. Test participants took 10 puffs over 3 minutes in each case (solid line (2^{nd} highest peak): cigarette, dark dotted line (highest peak): test formulation 1 (TF1-nicotine salt formulation), light dotted line: test formulation 2 (TF2—nicotine formulation). Comparison between cigarette, TF1, and TF2 is shown in FIG. 2.

It is clearly shown in FIG. 2 that the test formulation with nicotine levulinate (TF1) causes a faster rise in heart rate than just nicotine (TF2). Also, TF1 more closely resembles the rate of increase for a cigarette. Other salts were tried and also found to increase heart rate relative to a pure nicotine solution. Thus, other suitable nicotine salts that cause the similar effect may be used in accordance with the practice of the present invention. For example, other keto acids (alpha-keto acids, beta-keto acids, gamma-keto acids, and the like) such as pyruvic acid, oxaloacetic acid, acetoacetic acid, and the like. This experience of increased heart rate comparable to that of a traditional burned cigarette has not been demonstrated or identified in other electronic cigarette devices, nor has it been demonstrated or identified in low temperature tobacco vaporization devices that do not burn the tobacco, even when a nicotine salt was used (a solution of 20% (W/W) or more of nicotine salt) as an additive to the tobacco. Thus the results from this experiment are surprising and unexpected.

In addition, the data appears to correlate well with the previous findings shown in FIG. 2.

As previously noted in the Satisfaction Study, the nicotine salts formulations with acids having vapor pressures between 20-300 mmHg @ 200° C. provide more satisfaction than the rest, with the exception of the nicotine salt formulation made with pyruvic acid, which has a boiling point of 165° C., as noted in FIG. 3. Based on the findings herein, it was anticipated that these nicotine salt formulations having either: a Vapor Pressure between 20-300 mmHg @ 200° C., a Vapor Pressure >20 mmHg @ 200° C., a difference between boiling point and melting point of at least 50° C., and a boiling point greater than 160° C., and a melting point less than 160° C., a difference between boiling point and melting point of at least 50° C., and a boiling point greater than 160° C., and a melting point less than 160° C., a difference between boiling point and melting point of at least 50° C., and a boiling point at most 40° C. less than operating temperature, and a melting point at least 40° C. lower than operating temperature, or a combination thereof produce one or more of the following effects:

Tₘₐₓ—Time to maximum blood concentration: Based on the results established herein, a user of an e-cigarette comprising the nicotine salt formulation will experience a comparable rate of physical and emotional satisfaction from using a formulation comprising a mixture of nicotine salts prepared with an appropriate acid at least 1.2× to 3× faster than using a formulation comprising a freebase nicotine. As illustrated in FIG. 1: Nicotine from a nicotine salts formulation appears to generate a heartbeat that is nearly 1.2 times that of a normal heart rate for an individual approximately 40 seconds after the commencement of puffing; whereas the nicotine from a nicotine freebase formulation appears to generate a heartbeat that is nearly 1.2 times that of a normal heart rate for an individual approximately 110 seconds after the commencement of puffing; a 2.75× difference in time to achieve a comparable initial satisfaction level.

Again this would not be inconsistent with the data from FIG. 2, where the data illustrated that at approximately 120 seconds (2 minutes), the heart rate of test participants reached a maximum of 105-110 bpm with either a regular cigarette or a nicotine salt formulation (TF1); whereas those same participants heart rates only reached a maximum of approximately 86 bpm at approximately 7 minutes with a nicotine freebase formulation (TF2); also a difference in effect of 1.2 times greater with nicotine salts (and regular cigarettes) versus freebase nicotine.

Further, when considering peak satisfaction levels (achieved at approximately 120 seconds from the initiation of puffing (time=0) and looking at the slope of the line for a normalized heart rate, the approximate slope of those nicotine salt formulations that exceeded the freebase nicotine formulation range between 0.0054 hrₙ/sec and 0.0025 hrₙ/sec. By comparison, the slope of the line for the freebase nicotine formulation is about 0.002. This would suggest that the concentration of available nicotine will be delivered to the user at a rate that is between 1.25 and 2.7 times faster than a freebase formulation.

In another measure of performance; Cₘₐₓ—Maximum blood nicotine concentration; it is anticipated that similar rates of increase will be measured in blood nicotine concentration, as those illustrated above. That is, it was anticipated based on the findings herein, and unexpected based on the art known to date, that there would be comparable Cₘₐₓ between the common cigarette and certain nicotine salt formulations, but with a lower Cₘₐₓ in a freebase nicotine solution.

Similarly, anticipated based on the findings herein, and unexpected based on the art known to date, that certain nicotine salt formulations would have higher rate of nicotine uptake levels in the blood at early time periods. Indeed, Example 8 presents data for multiple salt formulations consistent with these predictions which were made based on the findings and tests noted herein, and unexpected compared to the art available to date.

### Example 7

Heart Rate Study of Nicotine Solutions Via e-Cigarette

Exemplary formulations of nicotine levulinate, nicotine benzoate, nicotine succinate, nicotine salicylate, nicotine malate, nicotine pyruvate, nicotine citrate, nicotine sorbate, nicotine laurate, nicotine freebase, and a control of propylene glycol are prepared as noted in Example 1 and are administered in the same fashion by an electronic cigarette to the same human subject. About 0.5 mL of each solution is loaded into an “eRoll” cartridge atomizer (joyetech.com) to be used in the study. The atomizer is then attached to an “eRoll” e-cigarette (same manufacturer). The operating temperature of the e-cigarette is from about 150° C. to about 250° C., or from about 180° C. to about 220° C.

Heart rate measurements are taken for 6 minutes; from 1 minute before start of puffing, for 3 minutes during puffing, and continuing until 2 minutes after end of puffing. The test participant takes 10 puffs over 3 minutes in each case. The base heart rate is the average heart rate over the first 1 minute before start of puffing. Heart rate after puffing started is averaged over 20-second intervals. Normalized heart rate is defined as the ratio between individual heart rate data point and the base heart rate. Final results are presented as normalized heart rate.

### Example 8

### Blood Plasma Testing

Blood plasma testing was conducted on three subjects (n=3). Eight test articles were used in this study: one reference cigarette and seven blends used in an e-cigarette device having an operating temperature of the e-cigarette from about 150° C. to about 250° C., or from about 180° C. to about 220° C. The reference cigarette was Pall Mall (New Zealand). Seven blends were tested in the e-cigarette: 2% free base, 2% benzoate, 4% benzoate, 2% citrate, 2% malate, 2% salicylate, and 2% succinate. Except for 2% succinate (n=1), all other blends have n=3. The seven blends were liquid formulations prepared as described in Example 1.

The concentration of nicotine in each of the formulations was confirmed using UV spectrophotometer (Cary 60, manufactured by Agilent). The sample solutions for UV analysis were made by dissolving 20 mg of each of the formulations in 20 mL 0.3% HCl in water. The sample solutions were then scanned in UV spectrophotometer and the characteristic nicotine peak at 259 nm was used to quantify nicotine in the sample against a standard solution of 19.8 μg/mL nicotine in the same diluent. The standard solution was prepared by first dissolving 19.8 mg nicotine in 10 mL 0.3% HCl in water followed by a 1:100 dilution with 0.3% HCl in water. Nicotine concentrations reported for all formulations were within the range of 95%-105% of the claimed concentrations

All subjects were able to consume 30-55 mg of the liquid formulation of each tested blend using the e-cigarette.

Literature results: C. Bullen et al, Tobacco Control 2010, 19:98-103. Cigarette (5 min adlib, n=9): Tₘₐₓ=14.3 (8.8-19.9), Cₘₐₓ=13.4 (6.5-20.3) 1.4% E-cig (5 min adlib, n=8): Tₘₐₓ=19.6 (4.9-34.2), Cₘₐₓ=1.3 (0.0-2.6). Nicorette Inhalator (20 mg/20 min, n=10): Tₘₐₓ=32.0 (18.7-45.3), Cₘₐₓ=2.1 (1.0-3.1)

Estimated Cₘₐₓ of 2% nicotine blends: Cₘₐₓ=Mass consumed*Strength*Bioavailability/(Vol of Distribution*Body Weight)=40 mg*2%*80%/(2.6 L/kg*75 kg)=3.3 ng/ml

Estimated Cₘₐₓ of 4% nicotine blends: Cₘₐₓ=Mass consumed*Strength*Bioavailability/(Vol of Distribution*Body Weight)=40 mg*4%*80%/(2.6 L/kg*75 kg)=6.6 ng/ml

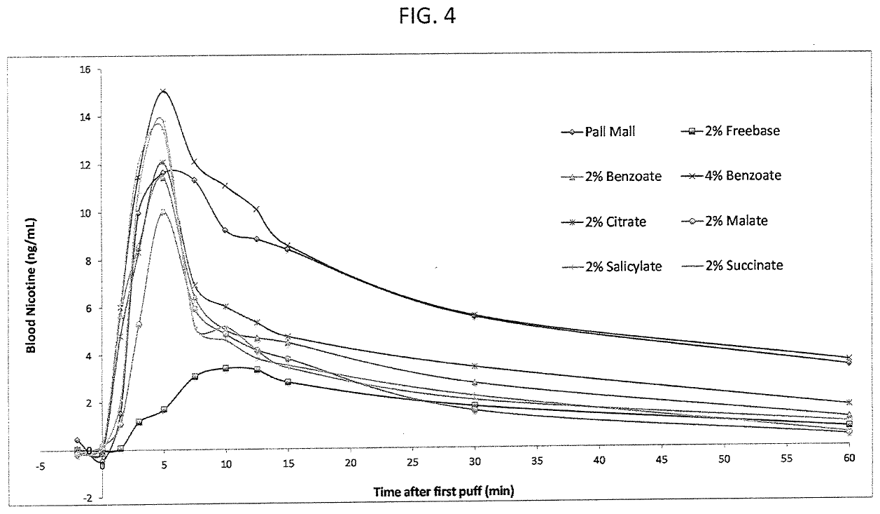

Pharmacokinetic profiles of the blood plasma testing are shown in FIG. 4; showing blood nicotine concentrations (ng/mL) over time after the first puff (inhalation) of the aerosol from the e-cigarette or the smoke of the Pall Mall. Ten puffs were taken at 30 sec intervals starting at time=0 and continuing for 4.5 minutes. For ease of reference and review of FIG. 4, at the 5-minute timepoint, the curves on the graph show from top to bottom (highest average blood nicotine concentration to lowest average blood nicotine concentration) are 4% benzoate, 2% succinate, 2% salicylate, 2% citrate, Pall Mall cigarette, 2% benzoate, 2% malate, and 2% free base blend. Although noted as highest to lowest at this time point, this is not to say that there is a statistically significant difference between any of the salt formulations, or between any of the salt formulations and the Pall Mall cigarette. However, it is possible there may be a statistically significant difference between the Cₘₐₓ of particular salt formulations, and it is also likely based on the data shown in FIG. 4 and in other studies herein that the freebase formulation is statistically different from salt formulations and/or the Pall Mall with respect to Cₘₐₓ, since it appears lower than others tested at several time points. One of skill in the art, upon review of the disclosure herein could properly power a test to determine actual statistically-based differences between one or more formulations and the cigarette, or between the formulations themselves in an e-cigarette. For ease of reference Tables 1 & 2 present the amount of nicotine detected (as an average of all users) for each formulation and the Pall Mall, presented in ng/ml, along with Cₘₐₓ and Tₘₐₓ and AUC. Data from these tables, along with the raw data therefore, was used to generate FIGS. 4-6.

TABLE 1

| | Pall | 2% | 2% | 4% |
|---|---|---|---|---|
| Time | Mall | Freebase | Benzoate | Benzoate |
| −2 | 0.46 | 0.03 | 0.09 | 0.05 |
| 0 | −0.46 | −0.03 | −0.09 | −0.05 |
| 1.5 | 1.54 | 0.08 | 5.67 | 6.02 |
| 3 | 9.98 | 1.19 | 8.60 | 11.47 |
| 5 | 11.65 | 1.70 | 11.44 | 15.06 |
| 7.5 | 11.34 | 3.09 | 6.43 | 12.12 |
| 10 | 9.24 | 3.42 | 5.03 | 11.08 |
| 12.5 | 8.85 | 3.35 | 4.68 | 10.10 |
| 15 | 8.40 | 2.81 | 4.47 | 8.57 |
| 30 | 5.51 | 1.74 | 2.72 | 5.56 |
| 60 | 3.39 | 0.79 | 1.19 | 3.60 |
| Tₘₐₓ (min) | 5.17 | 10.00 | 6.67 | 5.83 |
| Cₘₐₓ (ng/mL) | 11.65 | 3.42 | 11.44 | 15.06 |
| AUC (ng*min/mL) | 367.5 | 106.2 | 207.8 | 400.2 |

TABLE 2

| | 2% | 2% | 2% | 2% |
|---|---|---|---|---|
| Time | Citrate | Malate | Salicylate | Succinate |
| −2 | 0.06 | −0.17 | −0.19 | −0.06 |
| 0 | −0.06 | 0.17 | 0.19 | 0.06 |
| 1.5 | 4.80 | 1.09 | 6.14 | 2.10 |
| 3 | 8.33 | 5.30 | 12.04 | 10.81 |
| 5 | 12.09 | 10.02 | 13.46 | 13.81 |
| 7.5 | 6.93 | 5.93 | 5.21 | 5.15 |
| 10 | 6.01 | 4.85 | 4.60 | 5.18 |
| 12.5 | 5.34 | 4.17 | 3.83 | 4.17 |
| 15 | 4.72 | 3.79 | 3.52 | 3.41 |
| 30 | 3.40 | 1.56 | 2.19 | 2.01 |
| 60 | 1.70 | 0.46 | 0.55 | 1.00 |
| Tₘₐₓ (min) | 5.83 | 5.00 | 4.33 | 5.00 |
| Cₘₐₓ (ng/mL) | 12.09 | 10.02 | 13.46 | 13.81 |
| AUC (ng*min/mL) | 238.0 | 146.1 | 182.9 | 179.5 |

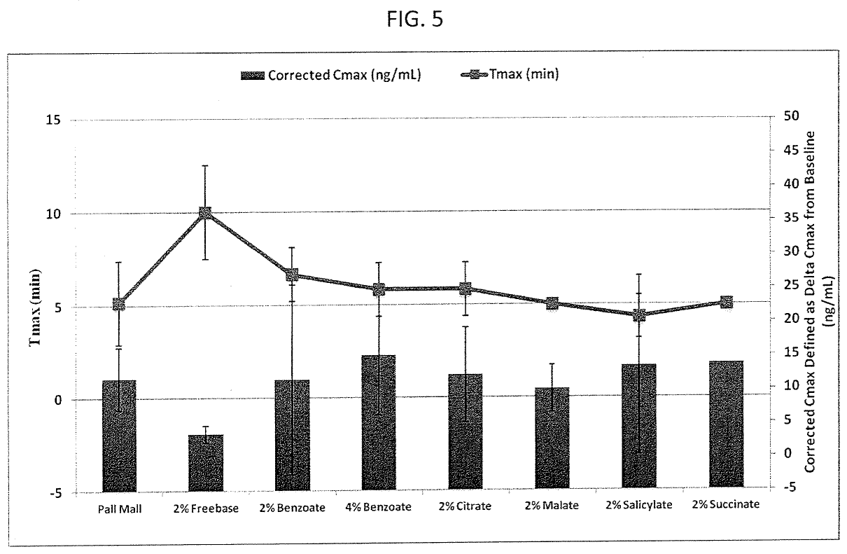

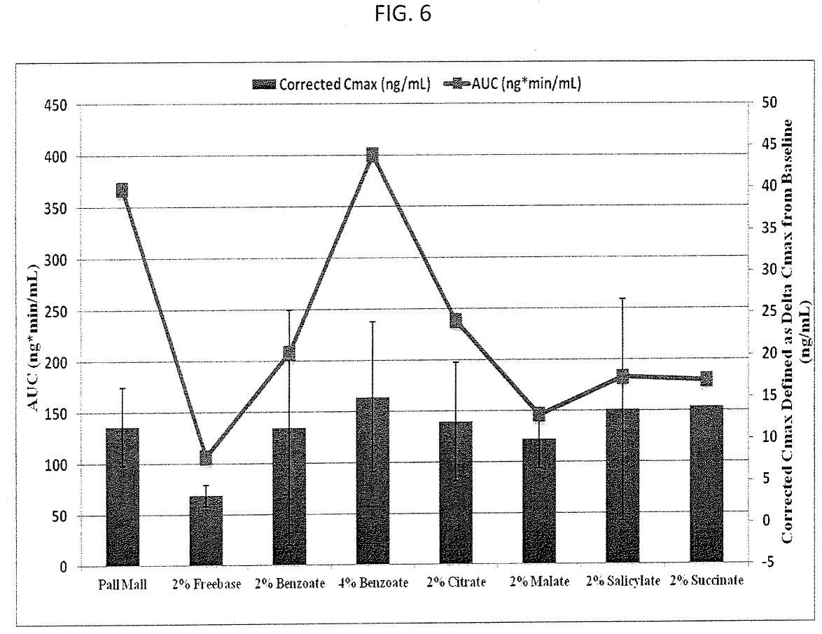

Comparison of Tₘₐₓ and Cₘₐₓ of the seven blends and reference cigarette are shown in FIG. 5. Comparison of Cₘₐₓ and AUC of the seven blends and reference cigarette are shown in FIG. 6. Due to the time limit of the wash-period, baseline blood nicotine concentration (at t=−2 and t=0 min) was higher for samples consumed at a later time on the test day. The data in FIGS. 4-6 show corrected blood nicotine concentration values (i.e. apparent blood nicotine concentration at each time point minus baseline nicotine concentration of the same sample).

Rates of nicotine uptake in the blood of the users of each sample within the first 90 seconds are shown in Table 3.

TABLE 3

| | Rate of |
|---|---|
| | nicotine |
| | uptake |
| Sample | (ng/mL/min) |
| 2% Salicylate | 4.09 |
| 2% Benzoate | 3.78 |
| 2% Citrate | 3.20 |
| 2% Succinate | 1.40 |
| Pall Mall (reference) | 1.03 |
| 2% Malate | 0.73 |
| 2% Freebase | 0.05 |
| 4% Benzoate | 4.01 |

Although the Tₘₐₓ and Cₘₐₓ values are comparable between the tested blends and the reference cigarette (with the exception of the 2% free base blend), the rates of nicotine absorption within the first 90 seconds differed among the test articles. In particular, four blends (2% salicylate, 2% benzoate, 4% benzoate, and 2% citrate) showed markedly higher rates of absorption within the first 90 seconds compared to the other blends and with the reference cigarette. These four blends contain salts (salicylate, benzoate, and citrate) which performed well in the Satisfaction Study of Example 3. Moreover, 2% benzoate and 4% benzoate had comparable rates of absorption, suggesting that a lower concentration of nicotinic salt may not adversely impact the rate of absorption.

### Example 9

### Blood Plasma Testing

Blood plasma testing is conducted on 24 subjects (n=24). Eight test articles are used in this study: one reference cigarette and seven blends delivered to a user in an e-cigarette as an aerosol. The operating temperature of the e-cigarette is from about 150° C. to about 250° C., or from about 180° C. to about 220° C. The reference cigarette is Pall Mall (New Zealand). Seven blends are tested: 2% free base, 2% benzoate, 4% benzoate, 2% citrate, 2% malate, 2% salicylate, and 2% succinate. The seven blends are liquid formulations prepared according to protocols similar to that described infra and in Example 1.

All subjects are to consume 30-55 mg of the liquid formulation of each tested blend. Ten puffs are to be taken at 30 sec intervals starting at time=0 and continuing for 4.5 minutes. Blood plasma testing is to occur for at least 60 minutes from the first puff (t=0) Pharmacokinetic data (e.g., Cₘₐₓ, Tₘₐₓ, AUC) for nicotine in the plasma of users are obtained at various time periods during those 60 minutes, along with rates of nicotine absorption within the first 90 seconds for each test article.

### Example 10

### Blood Plasma Testing

Blood plasma testing is conducted on twenty-four subjects (n=24). Eleven test articles are used in this study: one reference cigarette and ten blends delivered to a user in an e-cigarette as an aerosol. The reference cigarette is Pall Mall (New Zealand). The operating temperature of the e-cigarette is from about 150° C. to about 250° C., or from about 180° C. to about 220° C. Ten blends are tested: 2% free base, 2% benzoate, 2% sorbate, 2% pyruvate, 2% laurate, 2% levulinate, 2% citrate, 2% malate, 2% salicylate, and 2% succinate. The ten blends are liquid formulations prepared according to protocols similar to that described infra and in Example 1.

All subjects are to consume 30-55 mg of the liquid formulation of each tested blend. Ten puffs are to be taken at 30 sec intervals starting at time=0 and continuing for 4.5 minutes. Blood plasma testing is to occur for at least 60 minutes from the first puff (t=0). Pharmacokinetic data (e.g., Cₘₐₓ, Tₘₐₓ, AUC) for nicotine in the plasma of users are obtained at various time periods during those 60 minutes, along with rates of nicotine absorption within the first 90 seconds for each test article.

### Example 11

### Blood Plasma Testing

Blood plasma testing is conducted on twenty-four subjects (n=24). Twenty-one test articles are used in this study: one reference cigarette and twenty blends delivered to a user in an e-cigarette as an aerosol. The reference cigarette is Pall Mall (New Zealand). The operating temperature of the e-cigarette is from about 150° C. to about 250° C., or from about 180° C. to about 220° C. Twenty blends are tested: 2% free base, 4% free base, 2% benzoate, 4% benzoate, 2% sorbate, 4% sorbate, 2% pyruvate, 4% pyruvate, 2% laurate, 4% laurate, 2% levulinate, 4% levulinate, 2% citrate, 4% citrate, 2% malate, 4% malate, 2% salicylate, 4% salicylate, 2% succinate, and 4% succinate. The twenty blends are liquid formulations prepared according to protocols similar to that described infra and in Example 1.

All subjects are to consume 30-55 mg of the liquid formulation of each tested blend. Ten puffs are to be taken at 30 sec intervals starting at time=0 and continuing for 4.5 minutes. Blood plasma testing is to occur for at least 60 minutes from the first puff (t=0). Pharmacokinetic data (e.g., Cₘₐₓ, Tₘₐₓ, AUC) for nicotine in the plasma of users are obtained at various time periods during those 60 minutes, along with rates of nicotine absorption within the first 90 seconds for each test article.

### Example 12

### Blood Plasma Testing

Blood plasma testing is conducted on twenty-four subjects (n=24). Twenty-one test articles are used in this study: one reference cigarette and twenty blends delivered to a user in an e-cigarette as an aerosol. The reference cigarette is Pall Mall (New Zealand). The operating temperature of the e-cigarette is from about 150° C. to about 250° C., or from about 180° C. to about 220° C. Twenty blends are tested: 2% free base, 1% free base, 2% benzoate, 1% benzoate, 2% sorbate, 1% sorbate, 2% pyruvate, 1% pyruvate, 2% laurate, 1% laurate, 2% levulinate, 1% levulinate, 2% citrate, 1% citrate, 2% malate, 1% malate, 2% salicylate, 1% salicylate, 2% succinate, and 1% succinate. The twenty blends are liquid formulations prepared according to protocols similar to that described infra and in Example 1.

All subjects are to consume 30-55 mg of the liquid formulation of each tested blend. Ten puffs are to be taken at 30 sec intervals starting at time=0 and continuing for 4.5 minutes. Blood plasma testing is to occur for at least 60 minutes from the first puff (t=0). Pharmacokinetic data (e.g., Cₘₐₓ, Tₘₐₓ, AUC) for nicotine in the plasma of users are obtained at various time periods during those 60 minutes, along with rates of nicotine absorption within the first 90 seconds for each test article.

### EMBODIMENTS

Further understanding may be gained through contemplation of the numbered embodiments below.

Embodiment 1. A method of delivering nicotine to a user comprising operating an electronic cigarette to a user wherein the electronic cigarette comprises a nicotine salt formulation comprising a nicotine salt in a biologically acceptable liquid carrier wherein an acid used to form said nicotine salt is characterized by vapor pressure >20 mmHg at 200° C., and inhaling an aerosol generated from the nicotine salt formulation heated by the electronic cigarette.

Embodiment 2. A method of delivering nicotine to a user comprising operating an electronic cigarette to a user wherein the electronic cigarette comprises a nicotine salt formulation comprising a nicotine salt in a biologically acceptable liquid carrier wherein an acid used to form said nicotine salt is characterized by vapor pressure of about 20 to 200 mmHg at 200° C., and inhaling an aerosol generated from the nicotine salt formulation heated by the electronic cigarette.

Embodiment 3. A method of delivering nicotine to a user comprising operating an electronic cigarette wherein the electronic cigarette comprises a nicotine salt formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point, and inhaling an aerosol generated from the nicotine salt formulation heated by the electronic cigarette.

Embodiment 4. A method of delivering nicotine to a user comprising providing an electronic cigarette to a user wherein the electronic cigarette comprises a nicotine salt formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point, and inhaling an aerosol generated from the nicotine salt formulation heated by the electronic cigarette.

Embodiment 5. The method of any one of embodiments 1-3, wherein an operating temperature is from 150° C. to 250° C.

Embodiment 6. The method of any one of embodiments 1-3, wherein an operating temperature is from 180° C. to 220° C.

Embodiment 7. The method any one of embodiments 1-3, wherein an operating temperature is about 200° C.

Embodiment 8. The method of embodiment 4, wherein the operating temperature is from 150° C. to 250° C.

Embodiment 9. The method of embodiment 4, wherein the operating temperature is from 180° C. to 220° C.

Embodiment 10. The method of embodiment 4, wherein the operating temperature is about 200° C.

Embodiment 11. The method any one of embodiments 1-10, wherein the aerosol comprises condensate of the nicotine salt.

Embodiment 12. The method any one of embodiments 1-10, wherein the aerosol comprises condensate of freebase nicotine.

Embodiment 13. The method any one of embodiments 1-10, wherein the aerosol comprises condensate of freebase nicotine and condensate of the carrier.

Embodiment 14. The method any one of embodiments 1-10, wherein the aerosol comprises condensate of freebase nicotine and condensate of the acid.

Embodiment 15. The method any one of embodiments 1-14, wherein the aerosol comprises condensate in particle sizes from about 0.1 microns to about 5 microns.

Embodiment 16. The method any one of embodiments 1-14, wherein the aerosol comprises condensate in particle sizes from about 0.1 microns to about 1 or 2 microns.

Embodiment 17. The method any one of embodiments 1-14, wherein the aerosol comprises condensate in particle sizes from about 0.1 microns to about 0.7 microns.

Embodiment 18. The method any one of embodiments 1-14, wherein the aerosol comprises condensate in particle sizes from about 0.3 microns to about 0.4 microns.

Embodiment 19. The method any one of embodiments 1-18, wherein the acid is a carboxylic acid.

Embodiment 20. The method of any one of embodiments 1-18, wherein the acid used to form said nicotine salt is an organic acid.

Embodiment 21. The method of embodiment 20, wherein the organic acid is monocarboxylic acid, aromatic acid, or keto acid.

Embodiment 22. The method of embodiment 20, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 23. The method of any one of embodiments 1-18, wherein the acid used to form the nicotine salt is salicylic acid.

Embodiment 24. The method of any one of embodiments 1-18, wherein the acid used to form the nicotine salt is benzoic acid.

Embodiment 25. The method of any one of embodiments 1-18, wherein the acid used to form the nicotine salt is pyruvic acid.

Embodiment 26. The method of any one of embodiments 1-18, wherein the acid used to form the nicotine salt is sorbic acid.

Embodiment 27. The method of any one of embodiments 1-18, wherein the acid used to form the nicotine salt is lauric acid.

Embodiment 28. The method of any one of embodiments 1-18, wherein the acid used to form the nicotine salt is levulinic acid.

Embodiment 29. The method of any one of embodiments 1-18, wherein said nicotine salt comprises nicotine pyruvate.

Embodiment 30. The method of any one of embodiments 1-18, wherein said nicotine salt comprises nicotine salicylate.

Embodiment 31. The method of any one of embodiments 1-18, wherein said nicotine salt comprises nicotine sorbate.

Embodiment 32. The method of any one of embodiments 1-18, wherein said nicotine salt comprises nicotine laurate.

Embodiment 33. The method of any one of embodiments 1-18, wherein said nicotine salt comprises nicotine levulinate.

Embodiment 34. The method of any one of embodiments 1-18, wherein said nicotine salt comprises nicotine benzoate.

Embodiment 35. The method of any one of embodiments 1-34, wherein the liquid carrier comprises glycerol, propylene glycol, trimethylene glycol, water, ethanol or combinations thereof.

Embodiment 36. The method of any one of embodiments 1-34, wherein the liquid carrier comprises propylene glycol and vegetable glycerin.

Embodiment 37. The method of any one of embodiments 1-34, wherein the liquid carrier comprises 20% to 50% of propylene glycol and 80% to 50% of vegetable glycerin.

Embodiment 38. The method of any one of embodiments 1-34, wherein the liquid carrier comprises 30% propylene glycol and 70% vegetable glycerin.

Embodiment 39. The method of any one of embodiments 1-38, wherein the nicotine salt is in an amount that forms about 0.5% to about 20% nicotine in the inhalable aerosol.

Embodiment 40. The method of any one of embodiments 1-38, wherein the nicotine salt is in an amount that forms about 1% to about 20% nicotine in the inhalable aerosol.

Embodiment 41. The method of any one of embodiments 1-40, wherein the liquid formulation has a nicotine concentration of about 1% (w/w) to about 25% (w/w).

Embodiment 42. The method of any one of embodiments 1-40, wherein the liquid formulation has a nicotine concentration of about 1% (w/w) to about 20% (w/w).

Embodiment 43. The method of any one of embodiments 1-40, wherein the liquid formulation has a nicotine concentration of about 1% (w/w) to about 18% (w/w).

Embodiment 44. The method of any one of embodiments 1-40, wherein the liquid formulation has a nicotine concentration of about 1% (w/w) to about 15% (w/w).

Embodiment 45. The method of any one of embodiments 1-40, wherein the liquid formulation has a nicotine concentration of about 4% (w/w) to about 12% (w/w).

Embodiment 46. The method of any one of embodiments 1-40, wherein the liquid formulation has a nicotine concentration of about 4% (w/w).

Embodiment 47. The method of any one of embodiments 1-40, wherein the liquid formulation has a nicotine concentration of about 2% (w/w).

Embodiment 48. The method of any one of embodiments 1-47, wherein the formulation further comprises a flavorant.

Embodiment 49. The method of any one of embodiments 1-48, wherein the formulation is non-corrosive to an electronic cigarette.

Embodiment 50. The method of any one of embodiments 1-49, wherein the acid is stable at and below operating temperature or about 200° C.

Embodiment 51. The method of any one of embodiments 1-50, wherein the acid does not decompose at and below operating temperature or about 200° C.

Embodiment 52. The method of any one of embodiments 1-51, wherein the acid does not oxidize at and below operating temperature or about 200° C.

Embodiment 53. The method of any one of embodiments 1-52, wherein the formulation is non-corrosive to the electronic cigarette.

Embodiment 54. The method of any one of embodiments 1-53, wherein the formulation is non-toxic to a user of the electronic cigarette.

Embodiment 55. The method of any one of embodiments 1-54, wherein the formulation further comprises one or more additional nicotine salts in a biologically acceptable liquid carrier suitable for generating the inhalable aerosol upon heating.

Embodiment 56. The method of embodiment 55, wherein a second acid used to form the additional nicotine salt is selected from the group consisting of salicylic acid, sorbic acid, benzoic acid, pyruvic acid, lauric acid, and levulinic acid.

Embodiment 57. A method of delivering nicotine to the blood of a user, said method comprising providing an aerosol that is inhaled by the user from an electronic cigarette that comprises a nicotine salt formulation wherein providing the aerosol comprises the electronic cigarette heating the formulation thereby generating the aerosol, wherein the aerosol is effective in delivering a level of nicotine in the blood of the user that is at least 5 ng/ml at about 1.5 minutes after a first puff of ten puffs of the aerosol, each puff taken at 30 second intervals.

Embodiment 58. The method of embodiment 54, wherein the nicotine salt formulation comprises a nicotine salt in a biologically acceptable liquid carrier wherein an acid used to form said nicotine salt is characterized by vapor pressure >20 mmHg at 200° C.

Embodiment 59. The method of embodiment 54, wherein the nicotine salt formulation comprises a nicotine salt in a biologically acceptable liquid carrier wherein an acid used to form said nicotine salt is characterized by vapor pressure of about 20 to 200 mmHg at 200° C.

Embodiment 60. The method of embodiment 54, wherein the nicotine salt formulation comprises a nicotine salt in a biologically acceptable liquid carrier wherein an acid used to form said nicotine salt is further characterized by a melting point <160° C., a boiling point >160° C., and at least a 50-degree difference between the melting point and the boiling point.

Embodiment 61. The method of any one of embodiments 57-60, wherein the heating of the formulation is at a temperature from 150° C. to 250° C.

Embodiment 62. The method of any one of embodiments 57-60, wherein the heating of the formulation is at a temperature from 180° C. to 220° C.

Embodiment 63. The method of any one of embodiments 57-60, wherein the heating of the formulation is at a temperature of about 200° C.

Embodiment 64. The method of embodiment 54, wherein the nicotine salt formulation comprises a nicotine salt in a biologically acceptable liquid carrier wherein an acid used to form said nicotine salt is further characterized by a melting point at least 40 degrees lower than the operating temperature of the electronic cigarette, a boiling point no more than 40 degrees lower than the operating temperature of the electronic cigarette, and at least a 50-degree difference between the melting point and the boiling point, and the operating temperature is 200° C.

Embodiment 65. The method of any one of embodiments 57-64, wherein the Cmax is over 10 ng/ml on average.

Embodiment 66. The method of any one of embodiments 57-64, wherein the Cmax is over 11 ng/ml on average.

Embodiment 67. The method of any one of embodiments 57-64, wherein the Cmax is between 10 ng/mL and 16 ng/mL on average.

Embodiment 68. The method of any one of embodiments 57-64, wherein the Cmax is between 11 ng/ml and 15 ng/mL on average.

Embodiment 69. The method of any one of embodiments 57-64, wherein the Cmax is between 11 ng/ml and 14 ng/mL on average.

Embodiment 70. The method of any one of embodiments 57-69, wherein the Tmax under 10 minutes on average.

Embodiment 71. The method of any one of embodiments 57-69, wherein the Tmax is under 9 minutes on average.

Embodiment 72. The method of any one of embodiments 57-69, wherein the Tmax is under 8 minutes on average.

Embodiment 73. The method of any one of embodiments 57-69, wherein the Tmax is under 7 minutes on average.

Embodiment 74. The method of any one of embodiments 54-63, wherein the Tmax is from 3 minutes to 10 minutes on average.

Embodiment 75. The method of any one of embodiments 57-69, wherein the Tmax is from 3 minutes to 7.5 minutes on average.

Embodiment 76. The method of any one of embodiments 57-75, wherein the aerosol comprises condensate of the nicotine salt.

Embodiment 77. The method of any one of embodiments 57-75, wherein the aerosol comprises condensate of freebase nicotine.

Embodiment 78. The method of any one of embodiments 57-75, wherein the aerosol comprises condensate of freebase nicotine and condensate of the carrier.

Embodiment 79. The method of any one of embodiments 57-75, wherein the aerosol comprises condensate of freebase nicotine and condensate of the acid.

Embodiment 80. The method of any one of embodiments 57-79, wherein the aerosol comprises condensate in particle sizes from about 0.1 microns to about 5 microns.

Embodiment 81. The method of any one of embodiments 57-79, wherein the aerosol comprises condensate in particle sizes from about 0.1 microns to about 1 or 2 microns.

Embodiment 82. The method of any one of embodiments 57-79, wherein the aerosol comprises condensate in particle sizes from about 0.1 microns to about 0.7 microns.

Embodiment 83. The method of any one of embodiments 57-79, wherein the aerosol comprises condensate in particle sizes from about 0.3 microns to about 0.4 microns.

Embodiment 84. The method of any one of embodiments 57-83, wherein the acid is a carboxylic acid.

Embodiment 85. The method of any one of embodiments 57-83, wherein the acid used to form said nicotine salt is an organic acid.

Embodiment 86. The method of embodiment 85, wherein the organic acid is monocarboxylic acid, aromatic acid, or keto acid.

Embodiment 87. The method of embodiment 85, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 88. The method of any one of embodiments 57-83, wherein the acid used to form the nicotine salt is salicylic acid.

Embodiment 89. The method of any one of embodiments 57-83, wherein the acid used to form the nicotine salt is benzoic acid.

Embodiment 90. The method of any one of embodiments 57-83, wherein the acid used to form the nicotine salt is pyruvic acid.

Embodiment 91. The method of any one of embodiments 57-83, wherein the acid used to form the nicotine salt is sorbic acid.

Embodiment 92. The method of any one of embodiments 57-83, wherein the acid used to form the nicotine salt is lauric acid.

Embodiment 93. The method of any one of embodiments 57-83, wherein the acid used to form the nicotine salt is levulinic acid.

Embodiment 94. The method of any one of embodiments 57-83, wherein said nicotine salt comprises nicotine pyruvate.

Embodiment 95. The method of any one of embodiments 57-83, wherein said nicotine salt comprises nicotine salicylate.

Embodiment 96. The method of any one of embodiments 57-83, wherein said nicotine salt comprises nicotine sorbate.

Embodiment 97. The method of any one of embodiments 57-83, wherein said nicotine salt comprises nicotine laurate.

Embodiment 98. The method of any one of embodiments 57-83, wherein said nicotine salt comprises nicotine levulinate.

Embodiment 99. The method of any one of embodiments 57-83, wherein said nicotine salt comprises nicotine benzoate.

Embodiment 100. The method of any one of embodiments 57-99, wherein the liquid carrier comprises glycerol, propylene glycol, trimethylene glycol, water, ethanol or combinations thereof.

Embodiment 101. The method of any one of embodiments 57-99, wherein the liquid carrier comprises propylene glycol and vegetable glycerin.

Embodiment 102. The method of any one of embodiments 57-99, wherein the liquid carrier comprises 20% to 50% of propylene glycol and 80% to 50% of vegetable glycerin.

Embodiment 103. The method of any one of embodiments 57-99, wherein the liquid carrier comprises 30% propylene glycol and 70% vegetable glycerin.

Embodiment 104. The method of any one of embodiments 57-103, wherein the nicotine salt is in an amount that forms about 0.5% to about 20% nicotine in the inhalable aerosol.

Embodiment 105. The method of any one of embodiments 57-103, wherein the nicotine salt is in an amount that forms about 1% to about 20% nicotine in the inhalable aerosol.

Embodiment 106. The method of any one of embodiments 57-105, wherein the liquid formulation has a nicotine concentration of about 1% (w/w) to about 25% (w/w).

Embodiment 107. The method of any one of embodiments 57-105, wherein the liquid formulation has a nicotine concentration of about 1% (w/w) to about 20% (w/w).

Embodiment 108. The method of any one of embodiments 57-105, wherein the liquid formulation has a nicotine concentration of about 1% (w/w) to about 18% (w/w).

Embodiment 109. The method of any one of embodiments 57-105, wherein the liquid formulation has a nicotine concentration of about 1% (w/w) to about 15% (w/w).

Embodiment 110. The method of any one of embodiments 57-105, wherein the liquid formulation has a nicotine concentration of about 4% (w/w) to about 12% (w/w).

Embodiment 111. The method of any one of embodiments 57-105, wherein the liquid formulation has a nicotine concentration of about 4% (w/w).

Embodiment 112. The method of any one of embodiments 57-105, wherein the liquid formulation has a nicotine concentration of about 2% (w/w).

Embodiment 113. The method of any one of embodiments 57-112, wherein the formulation further comprises a flavorant.

Embodiment 114. The method of any one of embodiments 57-113, wherein the formulation is non-corrosive to an electronic cigarette.

Embodiment 115. The method of any one of embodiments 57-114, wherein the acid is stable at and below operating temperature or about 200° C.

Embodiment 116. The method of any one of embodiments 57-115, wherein the acid does not decompose at and below operating temperature or about 200° C.

Embodiment 117. The method of any one of embodiments 57-116, wherein the acid does not oxidize at and below operating temperature or about 200° C.

Embodiment 118. The method of any one of embodiments 57-117, wherein the formulation is non-corrosive to the electronic cigarette.

Embodiment 119. The method of any one of embodiments 57-118, wherein the formulation is non-toxic to a user of the electronic cigarette.

Embodiment 120. The method of any one of embodiments 57-119, wherein the formulation further comprises one or more additional nicotine salts in a biologically acceptable liquid carrier suitable for generating the inhalable aerosol upon heating.

Embodiment 121. The method of embodiment 120, wherein a second acid used to form the additional nicotine salt is selected from the group consisting of salicylic acid, sorbic acid, benzoic acid, pyruvic acid, lauric acid, and levulinic acid.

Embodiment 122. A nicotine salt liquid formulation in an electronic cigarette for generating an inhalable aerosol upon heating in the electronic cigarette, the formulation in the cigarette comprising a nicotine salt in a biologically acceptable liquid carrier wherein an acid used to form said nicotine salt is characterized by vapor pressure >20 mmHg at 200° C.

Embodiment 123. A nicotine salt liquid formulation in an electronic cigarette for generating an inhalable aerosol upon heating in the electronic cigarette, the formulation in the cigarette comprising a nicotine salt in a biologically acceptable liquid carrier wherein an acid used to form said nicotine salt is characterized by vapor pressure of about 20 to 200 mmHg at 200° C.

Embodiment 124. A nicotine salt liquid formulation in an electronic cigarette for generating an inhalable aerosol upon heating in the electronic cigarette, the formulation in the cigarette comprising a nicotine salt in a biologically acceptable liquid carrier wherein an acid used to form said nicotine salt is further characterized by a melting point <160° C., a boiling point >160° C., and at least a 50-degree difference between the melting point and the boiling point.

Embodiment 125. A nicotine salt liquid formulation in an electronic cigarette for generating an inhalable aerosol upon heating in the electronic cigarette, the formulation in the cigarette comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Embodiment 126. The nicotine salt liquid formulation in the electronic cigarette of any one of embodiments 122-124, wherein the electronic cigarette heats the nicotine salt formulation to an operating temperature from 150° C. to 250° C.

Embodiment 127. The nicotine salt liquid formulation in the electronic cigarette of any one of embodiments 122-124, wherein the electronic cigarette heats the nicotine salt formulation to an operating temperature from 180° C. to 220° C.

Embodiment 128. The nicotine salt liquid formulation in the electronic cigarette of any one of embodiments 122-124, wherein the electronic cigarette heats the nicotine salt formulation to an operating temperature of about 200° C.

Embodiment 129. The nicotine salt liquid formulation in the electronic cigarette of embodiment 125, wherein the operating temperature is from 150° C. to 250° C.

Embodiment 130. The nicotine salt liquid formulation in the electronic cigarette of embodiment 125, wherein the operating temperature is from 180° C. to 220° C.

Embodiment 131. The nicotine salt liquid formulation in the electronic cigarette of embodiment 125, wherein the operating temperature is about 200° C.

Embodiment 132. The nicotine salt liquid formulation in the electronic cigarette of any one of embodiments 122-131, wherein the acid is a carboxylic acid.

Embodiment 133. The nicotine salt liquid formulation in the electronic cigarette of any one of embodiments 122-131, wherein the acid used to form said nicotine salt is an organic acid.

Embodiment 134. The nicotine salt liquid formulation in the electronic cigarette of embodiment 133, wherein the organic acid is monocarboxylic acid, aromatic acid, or keto acid.

Embodiment 135. The nicotine salt liquid formulation in the electronic cigarette of embodiment 133, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid

Embodiment 136. The nicotine salt liquid formulation in the electronic cigarette of any one of embodiments 122-131, wherein the acid used to form the nicotine salt is salicylic acid.

Embodiment 137. The nicotine salt liquid formulation in the electronic cigarette of any one of embodiments 122-131, wherein the acid used to form the nicotine salt is benzoic acid.

Embodiment 138. The nicotine salt liquid formulation in the electronic cigarette of any one of embodiments 122-131, wherein the acid used to form the nicotine salt is pyruvic acid.

Embodiment 139. The nicotine salt liquid formulation in the electronic cigarette of any one of embodiments 122-131, wherein the acid used to form the nicotine salt is sorbic acid.

Embodiment 140. The nicotine salt liquid formulation in the electronic cigarette of any one of embodiments 122-131, wherein the acid used to form the nicotine salt is lauric acid.

Embodiment 141. The nicotine salt liquid formulation in the electronic cigarette of any one of embodiments 122-131, wherein the acid used to form the nicotine salt is levulinic acid.

Embodiment 142. The nicotine salt liquid formulation in the electronic cigarette of any one of embodiments 122-131, wherein said nicotine salt comprises nicotine pyruvate.

Embodiment 143. The nicotine salt liquid formulation in the electronic cigarette of any one of embodiments 122-131, wherein said nicotine salt comprises nicotine salicylate.

Embodiment 144. The nicotine salt liquid formulation in the electronic cigarette of any one of embodiments 122-131, wherein said nicotine salt comprises nicotine sorbate.

Embodiment 145. The nicotine salt liquid formulation in the electronic cigarette of any one of embodiments 122-131, wherein said nicotine salt comprises nicotine laurate.

Embodiment 146. The nicotine salt liquid formulation in the electronic cigarette of any one of embodiments 122-131, wherein said nicotine salt comprises nicotine levulinate.

Embodiment 147. The nicotine salt liquid formulation in the electronic cigarette of any one of embodiments 122-131, wherein said nicotine salt comprises nicotine benzoate.

Embodiment 148. The nicotine salt liquid formulation in the electronic cigarette of any one of embodiments 122-147, wherein the liquid carrier comprises glycerol, propylene glycol, trimethylene glycol, water, ethanol or combinations thereof.

Embodiment 149. The nicotine salt liquid formulation in the electronic cigarette of any one of embodiments 122-147, wherein the liquid carrier comprises propylene glycol and vegetable glycerin.

Embodiment 150. The nicotine salt liquid formulation in the electronic cigarette of any one of embodiments 122-147, wherein the liquid carrier comprises 20% to 50% of propylene glycol and 80% to 50% of vegetable glycerin.

Embodiment 151. The nicotine salt liquid formulation in the electronic cigarette of any one of embodiments 122-147, wherein the liquid carrier comprises 30% propylene glycol and 70% vegetable glycerin.

Embodiment 152. The nicotine salt liquid formulation in the electronic cigarette of any one of embodiments 122-151, wherein the nicotine salt is in an amount that forms about 0.5% to about 20% nicotine in the inhalable aerosol.

Embodiment 153. The nicotine salt liquid formulation in the electronic cigarette of any one of embodiments 122-151, wherein the nicotine salt is in an amount that forms about 1% to about 20% nicotine in the inhalable aerosol.

Embodiment 154. The nicotine salt liquid formulation in the electronic cigarette of any one of embodiments 122-153, wherein the liquid formulation has a nicotine concentration of about 1% (w/w) to about 25% (w/w).

Embodiment 155. The nicotine salt liquid formulation in the electronic cigarette of any one of embodiments 122-153, wherein the liquid formulation has a nicotine concentration of about 1% (w/w) to about 20% (w/w).

Embodiment 156. The nicotine salt liquid formulation in the electronic cigarette of any one of embodiments 122-153, wherein the liquid formulation has a nicotine concentration of about 1% (w/w) to about 18% (w/w).

Embodiment 157. The nicotine salt liquid formulation in the electronic cigarette of any one of embodiments 122-153, wherein the liquid formulation has a nicotine concentration of about 1% (w/w) to about 15% (w/w).

Embodiment 158. The nicotine salt liquid formulation in the electronic cigarette of any one of embodiments 122-153, wherein the liquid formulation has a nicotine concentration of about 4% (w/w) to about 12% (w/w).

Embodiment 159. The nicotine salt liquid formulation in the electronic cigarette of any one of embodiments 122-153, wherein the liquid formulation has a nicotine concentration of about 4% (w/w).

Embodiment 160. The nicotine salt liquid formulation in the electronic cigarette of any one of embodiments 122-153, wherein the liquid formulation has a nicotine concentration of about 2% (w/w).

Embodiment 161. The nicotine salt liquid formulation in the electronic cigarette of any one of embodiments 122-160, wherein the formulation further comprises a flavorant.

Embodiment 162. The nicotine salt liquid formulation in the electronic cigarette of any one of embodiments 122-161, wherein the formulation is non-corrosive to an electronic cigarette.

Embodiment 163. The nicotine salt liquid formulation in the electronic cigarette of any one of embodiments 122-162, wherein the acid is stable at and below operating temperature or about 200° C.

Embodiment 164. The nicotine salt liquid formulation in the electronic cigarette of any one of embodiments 122-163, wherein the acid does not decompose at and below operating temperature or about 200° C.

Embodiment 165. The nicotine salt liquid formulation in the electronic cigarette of any one of embodiments 122-164, wherein the acid does not oxidize at and below operating temperature or about 200° C.

Embodiment 166. The nicotine salt liquid formulation in the electronic cigarette of any one of embodiments 122-165, wherein the formulation is non-corrosive to the electronic cigarette.

Embodiment 167. The nicotine salt liquid formulation in the electronic cigarette of any one of embodiments 122-166, wherein the formulation is non-toxic to a user of the electronic cigarette.

Embodiment 168. The nicotine salt liquid formulation in the electronic cigarette of any one of embodiments 122-167 further comprising one or more additional nicotine salts in a biologically acceptable liquid carrier suitable for generating the inhalable aerosol upon heating.

Embodiment 169. The nicotine salt liquid formulation in the electronic cigarette of embodiment 168, wherein a second acid used to form the additional nicotine salt is selected from the group consisting of salicylic acid, sorbic acid, benzoic acid, pyruvic acid, lauric acid, and levulinic acid.

Embodiment 170. A nicotine salt liquid formulation for generating an inhalable aerosol upon heating in the electronic cigarette, the nicotine salt liquid formulation comprising a nicotine salt in a biologically acceptable liquid carrier wherein an acid used to form said nicotine salt is characterized by vapor pressure >20 mmHg at 200° C.

Embodiment 171. A nicotine salt liquid formulation for generating an inhalable aerosol upon heating in the electronic cigarette, the nicotine salt liquid formulation comprising a nicotine salt in a biologically acceptable liquid carrier wherein an acid used to form said nicotine salt is characterized by vapor pressure of about 20 to 200 mmHg at 200° C.

Embodiment 172. A nicotine salt liquid formulation for generating an inhalable aerosol upon heating in the electronic cigarette, the nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point.

Embodiment 173. A nicotine salt liquid formulation for generating an inhalable aerosol upon heating in the electronic cigarette, the nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Embodiment 174. The nicotine salt liquid formulation of any one of embodiments 170-172, wherein the electronic cigarette heats the nicotine salt formulation to an operating temperature from 150° C. to 250° C.

Embodiment 175. The nicotine salt liquid formulation of any one of embodiments 170-172, wherein the electronic cigarette heats the nicotine salt formulation to an operating temperature from 180° C. to 220° C.

Embodiment 176. The nicotine salt liquid formulation of any one of embodiments 170-172, wherein the electronic cigarette heats the nicotine salt formulation to an operating temperature of about 200° C.

Embodiment 177. The nicotine salt liquid formulation of embodiment 173, wherein the operating temperature is from 150° C. to 250° C.

Embodiment 178. The nicotine salt liquid formulation of embodiment 173, wherein the operating temperature is from 180° C. to 220° C.

Embodiment 179. The nicotine salt liquid formulation of embodiment 173, wherein the operating temperature is about 200° C.

Embodiment 180. The nicotine salt liquid formulation of any one of embodiments 170-179, wherein the acid is a carboxylic acid.

Embodiment 181. The nicotine salt liquid formulation of any one of embodiments 170-179, wherein the acid used to form said nicotine salt is an organic acid.

Embodiment 182. The nicotine salt liquid formulation of embodiment 181, wherein the organic acid is monocarboxylic acid, aromatic acid, or keto acid.

Embodiment 183. The nicotine salt liquid formulation of embodiment 181, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 184. The liquid formulation of any one of embodiments 170-179, wherein the acid used to form the nicotine salt is salicylic acid.

Embodiment 185. The nicotine salt liquid formulation of any one of embodiments 170-179, wherein the acid used to form the nicotine salt is benzoic acid.

Embodiment 186. The nicotine salt liquid formulation of any one of embodiments 170-179, wherein the acid used to form the nicotine salt is pyruvic acid.

Embodiment 187. The nicotine salt liquid formulation of any one of embodiments 170-179, wherein the acid used to form the nicotine salt is sorbic acid.

Embodiment 188. The nicotine salt liquid formulation of any one of embodiments 170-179, wherein the acid used to form the nicotine salt is lauric acid.

Embodiment 189. The nicotine salt liquid formulation of any one of embodiments 170-179, wherein the acid used to form the nicotine salt is levulinic acid.

Embodiment 190. The nicotine salt liquid formulation of any one of embodiments 170-179, wherein said nicotine salt comprises nicotine pyruvate.

Embodiment 191. The nicotine salt liquid formulation of any one of embodiments 170-179, wherein said nicotine salt comprises nicotine salicylate.

Embodiment 192. The nicotine salt liquid formulation of any one of embodiments 170-179, wherein said nicotine salt comprises nicotine sorbate.

Embodiment 193. The nicotine salt liquid formulation of any one of embodiments 170-179, wherein said nicotine salt comprises nicotine laurate.

Embodiment 194. The nicotine salt liquid formulation of any one of embodiments 170-179, wherein said nicotine salt comprises nicotine levulinate.

Embodiment 195. The nicotine salt liquid formulation of any one of embodiments 170-179, wherein said nicotine salt comprises nicotine benzoate.

Embodiment 196. The nicotine salt liquid formulation of any one of embodiments 170-195, wherein the liquid carrier comprises glycerol, propylene glycol, trimethylene glycol, water, ethanol or combinations thereof.

Embodiment 197. The nicotine salt liquid formulation of any one of embodiments 170-195, wherein the liquid carrier comprises propylene glycol and vegetable glycerin.

Embodiment 198. The nicotine salt liquid formulation of any one of embodiments 170-195, wherein the liquid carrier comprises 20% to 50% of propylene glycol and 80% to 50% of vegetable glycerin.

Embodiment 199. The nicotine salt liquid formulation of any one of embodiments 170-195, wherein the liquid carrier comprises 30% propylene glycol and 70% vegetable glycerin.

Embodiment 200. The nicotine salt liquid formulation of any one of embodiments 170-199, wherein the nicotine salt is in an amount that forms about 0.5% to about 20% nicotine in the inhalable aerosol.

Embodiment 201. The nicotine salt liquid formulation of any one of embodiments 170-199, wherein the nicotine salt is in an amount that forms about 1% to about 20% nicotine in the inhalable aerosol.

Embodiment 202. The nicotine salt liquid formulation of any one of embodiments 170-201, wherein the liquid formulation has a nicotine concentration of about 1% (w/w) to about 25% (w/w).

Embodiment 203. The nicotine salt liquid formulation of any one of embodiments 170-201, wherein the liquid formulation has a nicotine concentration of about 1% (w/w) to about 20% (w/w).

Embodiment 204. The nicotine salt liquid formulation of any one of embodiments 170-201, wherein the liquid formulation has a nicotine concentration of about 1% (w/w) to about 18% (w/w).

Embodiment 205. The nicotine salt liquid formulation of any one of embodiments 170-201, wherein the liquid formulation has a nicotine concentration of about 1% (w/w) to about 15% (w/w).

Embodiment 206. The nicotine salt liquid formulation of any one of embodiments 170-201, wherein the liquid formulation has a nicotine concentration of about 4% (w/w) to about 12% (w/w).

Embodiment 207. The nicotine salt liquid formulation of any one of embodiments 170-201, wherein the liquid formulation has a nicotine concentration of about 4% (w/w).

Embodiment 208. The nicotine salt liquid formulation of any one of embodiments 170-201, wherein the liquid formulation has a nicotine concentration of about 2% (w/w).

Embodiment 209. The nicotine salt liquid formulation of any one of embodiments 170-208, wherein the formulation further comprises a flavorant.

Embodiment 210. The nicotine salt liquid formulation of any one of embodiments 170-209, wherein the formulation is non-corrosive to an electronic cigarette.

Embodiment 211. The nicotine salt liquid formulation of any one of embodiments 170-210, wherein the acid is stable at and below operating temperature or about 200° C.

Embodiment 212. The nicotine salt liquid formulation of any one of embodiments 170-211, wherein the acid does not decompose at and below operating temperature or about 200° C.

Embodiment 213. The nicotine salt liquid formulation of any one of embodiments 170-212, wherein the acid does not oxidize at and below operating temperature or about 200° C.

Embodiment 214. The nicotine salt liquid formulation of any one of embodiments 170-213, wherein the formulation is non-corrosive to the electronic cigarette.

Embodiment 215. The nicotine salt liquid formulation of any one of embodiments 170-214, wherein the formulation is non-toxic to a user of the electronic cigarette.

Embodiment 216. The nicotine salt liquid formulation of any one of embodiments 170-215, further comprising one or more additional nicotine salts in a biologically acceptable liquid carrier suitable for generating the inhalable aerosol upon heating.

Embodiment 217. The nicotine salt liquid formulation of embodiment 216, wherein a second acid used to form the additional nicotine salt is selected from the group consisting of salicylic acid, sorbic acid, benzoic acid, pyruvic acid, lauric acid, and levulinic acid.

Embodiment 218. A nicotine salt liquid formulation for use in an electronic cigarette the nicotine salt liquid formulation comprising a nicotine salt in a biologically acceptable liquid carrier wherein an acid used to form said nicotine salt is characterized by vapor pressure >20 mmHg at 200° C.

Embodiment 219. A nicotine salt liquid formulation for use in an electronic cigarette the nicotine salt liquid formulation comprising a nicotine salt in a biologically acceptable liquid carrier wherein an acid used to form said nicotine salt is characterized by vapor pressure of about 20 to 200 mmHg at 200° C.

Embodiment 220. A nicotine salt liquid formulation for use in an electronic cigarette the nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point.

Embodiment 221. A nicotine salt liquid formulation for use in an electronic cigarette the nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Embodiment 222. The nicotine salt liquid formulation of any one of embodiments 218-220, wherein the electronic cigarette heats the nicotine salt formulation to an operating temperature from 150° C. to 250° C.

Embodiment 223. The nicotine salt liquid formulation of any one of embodiments 218-220, wherein the electronic cigarette heats the nicotine salt formulation to an operating temperature from 180° C. to 220° C.

Embodiment 224. The nicotine salt liquid formulation of any one of embodiments 218-220, wherein the electronic cigarette heats the nicotine salt formulation to an operating temperature of about 200° C.

Embodiment 225. The nicotine salt liquid formulation of embodiment 221, wherein the operating temperature is from 150° C. to 250° C.

Embodiment 226. The nicotine salt liquid formulation of embodiment 221, wherein the operating temperature is from 180° C. to 220° C.

Embodiment 227. The nicotine salt liquid formulation of embodiment 221, wherein the operating temperature is about 200° C.

Embodiment 228. The nicotine salt liquid formulation of any one of embodiments 218-227, wherein the acid is a carboxylic acid.

Embodiment 229. The nicotine salt liquid formulation of any one of embodiments 218-227, wherein the acid used to form said nicotine salt is an organic acid.

Embodiment 230. The nicotine salt liquid formulation of embodiment 229, wherein the organic acid is monocarboxylic acid, aromatic acid, or keto acid.

Embodiment 231. The nicotine salt liquid formulation of embodiment 229, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 232. The liquid formulation of any one of embodiments 218-227, wherein the acid used to form the nicotine salt is salicylic acid.

Embodiment 233. The nicotine salt liquid formulation of any one of embodiments 218-227, wherein the acid used to form the nicotine salt is benzoic acid.

Embodiment 234. The nicotine salt liquid formulation of any one of embodiments 218-227, wherein the acid used to form the nicotine salt is pyruvic acid.

Embodiment 235. The nicotine salt liquid formulation of any one of embodiments 218-227, wherein the acid used to form the nicotine salt is sorbic acid.

Embodiment 236. The nicotine salt liquid formulation of any one of embodiments 218-227, wherein the acid used to form the nicotine salt is lauric acid.

Embodiment 237. The nicotine salt liquid formulation of any one of embodiments 218-227, wherein the acid used to form the nicotine salt is levulinic acid.

Embodiment 238. The nicotine salt liquid formulation of any one of embodiments 218-227, wherein said nicotine salt comprises nicotine pyruvate.

Embodiment 239. The nicotine salt liquid formulation of any one of embodiments 218-227, wherein said nicotine salt comprises nicotine salicylate.

Embodiment 240. The nicotine salt liquid formulation of any one of embodiments 218-227, wherein said nicotine salt comprises nicotine sorbate.

Embodiment 241. The nicotine salt liquid formulation of any one of embodiments 218-227, wherein said nicotine salt comprises nicotine laurate.

Embodiment 242. The nicotine salt liquid formulation of any one of embodiments 218-227, wherein said nicotine salt comprises nicotine levulinate.

Embodiment 243. The nicotine salt liquid formulation of any one of embodiments 218-227, wherein said nicotine salt comprises nicotine benzoate.

Embodiment 244. The nicotine salt liquid formulation of any one of embodiments 218-243, wherein the liquid carrier comprises glycerol, propylene glycol, trimethylene glycol, water, ethanol or combinations thereof.

Embodiment 245. The nicotine salt liquid formulation of any one of embodiments 218-243, wherein the liquid carrier comprises propylene glycol and vegetable glycerin.

Embodiment 246. The nicotine salt liquid formulation of any one of embodiments 218-243, wherein the liquid carrier comprises 20% to 50% of propylene glycol and 80% to 50% of vegetable glycerin.

Embodiment 247. The nicotine salt liquid formulation of any one of embodiments 218-243, wherein the liquid carrier comprises 30% propylene glycol and 70% vegetable glycerin.

Embodiment 248. The nicotine salt liquid formulation of any one of embodiments 218-247, wherein the nicotine salt is in an amount that forms about 0.5% to about 20% nicotine in the inhalable aerosol.

Embodiment 249. The nicotine salt liquid formulation of any one of embodiments 218-247, wherein the nicotine salt is in an amount that forms about 1% to about 20% nicotine in the inhalable aerosol.

Embodiment 250. The nicotine salt liquid formulation of any one of embodiments 218-247, wherein the liquid formulation has a nicotine concentration of about 1% (w/w) to about 25% (w/w).

Embodiment 251. The nicotine salt liquid formulation of any one of embodiments 218-247, wherein the liquid formulation has a nicotine concentration of about 1% (w/w) to about 20% (w/w).

Embodiment 252. The nicotine salt liquid formulation of any one of embodiments 218-247, wherein the liquid formulation has a nicotine concentration of about 1% (w/w) to about 18% (w/w).

Embodiment 253. The nicotine salt liquid formulation of any one of embodiments 218-247, wherein the liquid formulation has a nicotine concentration of about 1% (w/w) to about 15% (w/w).

Embodiment 254. The nicotine salt liquid formulation of any one of embodiments 218-247, wherein the liquid formulation has a nicotine concentration of about 4% (w/w) to about 12% (w/w).

Embodiment 255. The nicotine salt liquid formulation of any one of embodiments 218-247, wherein the liquid formulation has a nicotine concentration of about 4% (w/w).

Embodiment 256. The nicotine salt liquid formulation of any one of embodiments 218-247, wherein the liquid formulation has a nicotine concentration of about 2% (w/w).

Embodiment 257. The nicotine salt liquid formulation of any one of embodiments 218-256, wherein the formulation further comprises a flavorant.

Embodiment 258. The nicotine salt liquid formulation of any one of embodiments 218-257, wherein the formulation is non-corrosive to an electronic cigarette.

Embodiment 259. The nicotine salt liquid formulation of any one of embodiments 218-258, wherein the acid is stable at and below operating temperature or about 200° C.

Embodiment 260. The nicotine salt liquid formulation of any one of embodiments 218-259, wherein the acid does not decompose at and below operating temperature or about 200° C.

Embodiment 261. The nicotine salt liquid formulation of any one of embodiments 218-260, wherein the acid does not oxidize at and below operating temperature or about 200° C.

Embodiment 262. The nicotine salt liquid formulation of any one of embodiments 218-261, wherein the formulation is non-corrosive to the electronic cigarette.

Embodiment 263. The nicotine salt liquid formulation of any one of embodiments 218-262, wherein the formulation is non-toxic to a user of the electronic cigarette.

Embodiment 264. The nicotine salt liquid formulation of any one of embodiments 218-263, further comprising one or more additional nicotine salts in a biologically acceptable liquid carrier suitable for generating the inhalable aerosol upon heating.

Embodiment 265. The nicotine salt liquid formulation of embodiment 264, wherein a second acid used to form the additional nicotine salt is selected from the group consisting of salicylic acid, sorbic acid, benzoic acid, pyruvic acid, lauric acid, and levulinic acid.

Embodiment 266. Use of a nicotine salt formulation for delivery of nicotine to a user from an electronic cigarette wherein the nicotine salt formulation comprises a nicotine salt in a biologically acceptable liquid carrier wherein an acid used to form said nicotine salt is characterized by vapor pressure >20 mmHg at 200° C., and the nicotine salt formulation is heated by the electronic cigarette to generate an aerosol inhalable by the user.

Embodiment 267. Use of a nicotine salt formulation for delivery of nicotine to a user from an electronic cigarette wherein the nicotine salt formulation comprises a nicotine salt in a biologically acceptable liquid carrier wherein an acid used to form said nicotine salt is characterized by vapor pressure of about 20 to 200 mmHg at 200° C., and the nicotine salt formulation is heated by the electronic cigarette to generate an aerosol inhalable by the user.

Embodiment 268. Use of a nicotine salt formulation for delivery of nicotine to a user from an electronic cigarette wherein the nicotine salt formulation comprises a nicotine salt in a biologically acceptable liquid carrier wherein an acid used to form said nicotine salt is further characterized by a melting point <160° C., a boiling point >160° C., and at least a 50-degree difference between the melting point and the boiling point, and the nicotine salt formulation is heated by the electronic cigarette to generate an aerosol inhalable by the user.

Embodiment 269. Use of a nicotine salt formulation for delivery of nicotine to the blood of a user from an electronic cigarette, wherein the nicotine salt formulation in the electronic cigarette is heated to form an aerosol which delivers a level of nicotine in the blood of the user that is at least 5 ng/mL at about 1.5 minutes after a first puff of ten puffs of the aerosol, each puff taken at 30 second intervals.

Embodiment 270. Use of a nicotine salt formulation for delivery of nicotine to a user from an electronic cigarette wherein the nicotine salt formulation comprises a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point, and the nicotine salt formulation is heated by the electronic cigarette to generate an aerosol inhalable by the user.

Embodiment 271. The use of any one of embodiments 266-269, wherein the electronic cigarette heats the nicotine salt formulation to an operating temperature is from 150° C. to 250° C.

Embodiment 272. The use of any one of embodiments 266-269, wherein the electronic cigarette heats the nicotine salt formulation to an operating temperature is from 180° C. to 220° C.

Embodiment 273. The use of any one of embodiments 266-269, wherein the electronic cigarette heats the nicotine salt formulation to an operating temperature is about 200° C.

Embodiment 274. The use of embodiment 270, wherein the operating temperature is from 150° C. to 250° C.

Embodiment 275. The use of embodiment 270, wherein the operating temperature is from 180° C. to 220° C.

Embodiment 276. The use of embodiment 270, wherein the operating temperature is about 200° C.

Embodiment 277. The use of any one of embodiments 266-276, wherein the aerosol comprises condensate of the nicotine salt.

Embodiment 278. The use of any one of embodiments 266-276, wherein the aerosol comprises condensate of freebase nicotine.

Embodiment 279. The use of any one of embodiments 266-276, wherein the aerosol comprises condensate of freebase nicotine and condensate of the carrier.

Embodiment 280. The use of any one of embodiments 266-276, wherein the aerosol comprises condensate of freebase nicotine and condensate of the acid.

Embodiment 281. The use of any one of embodiments 266-280, wherein the aerosol comprises condensate in particle sizes from about 0.1 microns to about 5 microns.

Embodiment 282. use of any one of embodiments 266-280, wherein the aerosol comprises condensate in particle sizes from about 0.1 microns to about 1 or 2 microns.

Embodiment 283. The use of any one of embodiments 266-280, wherein the aerosol comprises condensate in particle sizes from about 0.1 microns to about 0.7 microns.

Embodiment 284. The use of any one of embodiments 266-280, wherein the aerosol comprises condensate in particle sizes from about 0.3 microns to about 0.4 microns.

Embodiment 285. The use of any one of embodiments 266-284, wherein the acid is a carboxylic acid.

Embodiment 286. The use of any one of embodiments 266-284, wherein the acid used to form said nicotine salt is an organic acid.

Embodiment 287. The use of embodiment 286, wherein the organic acid is monocarboxylic acid, aromatic acid, or keto acid.

Embodiment 288. The use of embodiment 286, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 289. The use of any one of embodiments 266-284, wherein the acid used to form the nicotine salt is salicylic acid.

Embodiment 290. The use of any one of embodiments 266-284, wherein the acid used to form the nicotine salt is benzoic acid.

Embodiment 291. The use of any one of embodiments 266-284, wherein the acid used to form the nicotine salt is pyruvic acid.

Embodiment 292. The use of any one of embodiments 266-284, wherein the acid used to form the nicotine salt is sorbic acid.

Embodiment 293. The use of any one of embodiments 266-284, wherein the acid used to form the nicotine salt is lauric acid.

Embodiment 294. The use of any one of embodiments 266-284, wherein the acid used to form the nicotine salt is levulinic acid.

Embodiment 295. The use of any one of embodiments 266-284, wherein said nicotine salt comprises nicotine pyruvate.

Embodiment 296. The use of any one of embodiments 266-284, wherein said nicotine salt comprises nicotine salicylate.

Embodiment 297. The use of any one of embodiments 266-284, wherein said nicotine salt comprises nicotine sorbate.

Embodiment 298. The use of any one of embodiments 266-284, wherein said nicotine salt comprises nicotine laurate.

Embodiment 299. The use of any one of embodiments 266-284, wherein said nicotine salt comprises nicotine levulinate.

Embodiment 300. The use of any one of embodiments 266-284, wherein said nicotine salt comprises nicotine benzoate.

Embodiment 301. The use of any one of embodiments 266-300, wherein the liquid carrier comprises glycerol, propylene glycol, trimethylene glycol, water, ethanol or combinations thereof.

Embodiment 302. The use of any one of embodiments 266-300, wherein the liquid carrier comprises propylene glycol and vegetable glycerin.

Embodiment 303. The use of any one of embodiments 266-300, wherein the liquid carrier comprises 20% to 50% of propylene glycol and 80% to 50% of vegetable glycerin.

Embodiment 304. The use of any one of embodiments 266-300, wherein the liquid carrier comprises 30% propylene glycol and 70% vegetable glycerin.

Embodiment 305. The use of any one of embodiments 266-304, wherein the nicotine salt is in an amount that forms about 0.5% to about 20% nicotine in the inhalable aerosol.

Embodiment 306. The use of any one of embodiments 266-304, wherein the nicotine salt is in an amount that forms about 1% to about 20% nicotine in the inhalable aerosol.

Embodiment 307. The use of any one of embodiments 266-306, wherein the liquid formulation has a nicotine concentration of about 1% (w/w) to about 25% (w/w).

Embodiment 308. The use of any one of embodiments 266-306, wherein the liquid formulation has a nicotine concentration of about 1% (w/w) to about 20% (w/w).

Embodiment 309. The use of any one of embodiments 266-306, wherein the liquid formulation has a nicotine concentration of about 1% (w/w) to about 18% (w/w).

Embodiment 310. The use of any one of embodiments 266-306, wherein the liquid formulation has a nicotine concentration of about 1% (w/w) to about 15% (w/w).

Embodiment 311. The use of any one of embodiments 266-306, wherein the liquid formulation has a nicotine concentration of about 4% (w/w) to about 12% (w/w).

Embodiment 312. The use of any one of embodiments 266-306, wherein the liquid formulation has a nicotine concentration of about 4% (w/w).

Embodiment 313. The use of any one of embodiments 266-306, wherein the liquid formulation has a nicotine concentration of about 2% (w/w).

Embodiment 314. The use of any one of embodiments 266-313, wherein the formulation further comprises a flavorant.

Embodiment 315. The use of any one of embodiments 266-314, wherein the formulation is non-corrosive to an electronic cigarette.

Embodiment 316. The use of any one of embodiments 266-315, wherein the acid is stable at and below operating temperature or about 200° C.

Embodiment 317. The use of any one of embodiments 266-316, wherein the acid does not decompose at and below operating temperature or about 200° C.

Embodiment 318. The use of any one of embodiments 266-317, wherein the acid does not oxidize at and below operating temperature or about 200° C.

Embodiment 319. The use of any one of embodiments 266-318, wherein the formulation is non-corrosive to the electronic cigarette.

Embodiment 320. The use of any one of embodiments 266-319, wherein the formulation is non-toxic to a user of the electronic cigarette.

Embodiment 321. The use of any one of embodiments 266-320, wherein the formulation further comprises one or more additional nicotine salts in a biologically acceptable liquid carrier suitable for generating the inhalable aerosol upon heating.

Embodiment 322. The use of embodiment 321, wherein a second acid used to form the additional nicotine salt is selected from the group consisting of salicylic acid, sorbic acid, benzoic acid, pyruvic acid, lauric acid, and levulinic acid.

Embodiment 323. A cartomizer for an electronic cigarette comprising a nicotine salt liquid formulation comprising a nicotine salt in a biologically acceptable liquid carrier wherein an acid used to form said nicotine salt is characterized by vapor pressure >20 mmHg at 200° C., an atomizer comprising a heating element in fluid communication with the nicotine salt liquid formulation, and a fluid storage compartment that stores the nicotine salt liquid formulation.

Embodiment 324. A cartomizer for an electronic cigarette comprising a nicotine salt liquid formulation comprising a nicotine salt in a biologically acceptable liquid carrier wherein an acid used to form said nicotine salt is characterized by vapor pressure of about 20 to 200 mmHg at 200° C., an atomizer comprising a heating element in fluid communication with the nicotine salt liquid formulation, and a fluid storage compartment that stores the nicotine salt liquid formulation.

Embodiment 325. A cartomizer for an electronic cigarette comprising a nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point, an atomizer comprising a heating element in fluid communication with the nicotine salt liquid formulation, and a fluid storage compartment that stores the nicotine salt liquid formulation.

Embodiment 326. A cartomizer for an electronic cigarette comprising a nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point, an atomizer comprising a heating element in fluid communication with the nicotine salt liquid formulation, and a fluid storage compartment that stores the nicotine salt liquid formulation.

Embodiment 327. The cartomizer of any one of embodiments 323-325, wherein the electronic cigarette heats the nicotine salt formulation to an operating temperature from 150° C. to 250° C.

Embodiment 328. The cartomizer of any one of embodiments 323-325, wherein the electronic cigarette heats the nicotine salt formulation to an operating temperature from 180° C. to 220° C.

Embodiment 329. The cartomizer of any one of embodiments 323-325, wherein the electronic cigarette heats the nicotine salt formulation to an operating temperature of about 200° C.

Embodiment 330. The cartomizer of embodiment 326, wherein the operating temperature is from 150° C. to 250° C.

Embodiment 331. The cartomizer of embodiment 326, wherein the operating temperature is from 180° C. to 220° C.

Embodiment 332. The cartomizer of embodiment 326, wherein the operating temperature is about 200° C.

Embodiment 333. The cartomizer of any one of embodiments 323-332, wherein the cartomizer further comprises a mouthpiece.

Embodiment 334. The cartomizer of any one of embodiments 323-333, wherein the acid is a carboxylic acid.

Embodiment 335. The cartomizer of any one of embodiments 323-333, wherein the acid used to form said nicotine salt is an organic acid.

Embodiment 336. The cartomizer of embodiment 335, wherein the organic acid is monocarboxylic acid, aromatic acid, or keto acid.

Embodiment 337. The cartomizer of embodiment 335, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 338. The cartomizer of any one of embodiments 323-333, wherein the acid used to form the nicotine salt is salicylic acid.

Embodiment 339. The cartomizer of any one of embodiments 323-333, wherein the acid used to form the nicotine salt is benzoic acid.

Embodiment 340. The cartomizer of any one of embodiments 323-333, wherein the acid used to form the nicotine salt is pyruvic acid.

Embodiment 341. The cartomizer of any one of embodiments 323-333, wherein the acid used to form the nicotine salt is sorbic acid.

Embodiment 342. The cartomizer of any one of embodiments 323-333, wherein the acid used to form the nicotine salt is lauric acid.

Embodiment 343. The cartomizer of any one of embodiments 323-333, wherein the acid used to form the nicotine salt is levulinic acid.

Embodiment 344. The cartomizer of any one of embodiments 323-333, wherein said nicotine salt comprises nicotine pyruvate.

Embodiment 345. The cartomizer of any one of embodiments 323-333, wherein said nicotine salt comprises nicotine salicylate.

Embodiment 346. The cartomizer of any one of embodiments 323-333, wherein said nicotine salt comprises nicotine sorbate.

Embodiment 347. The cartomizer of any one of embodiments 323-333, wherein said nicotine salt comprises nicotine laurate.

Embodiment 348. The cartomizer of any one of embodiments 323-333, wherein said nicotine salt comprises nicotine levulinate.

Embodiment 349. The cartomizer of any one of embodiments 323-333, wherein said nicotine salt comprises nicotine benzoate.

Embodiment 350. The cartomizer of any one of embodiments 323-349, wherein the liquid carrier comprises glycerol, propylene glycol, trimethylene glycol, water, ethanol or combinations thereof.

Embodiment 351. The cartomizer of any one of embodiments 323-349, wherein the liquid carrier comprises propylene glycol and vegetable glycerin.

Embodiment 352. The cartomizer of any one of embodiments 323-349, wherein the liquid carrier comprises 20% to 50% of propylene glycol and 80% to 50% of vegetable glycerin.

Embodiment 353. The cartomizer of any one of embodiments 323-349, wherein the liquid carrier comprises 30% propylene glycol and 70% vegetable glycerin.

Embodiment 354. The cartomizer of any one of embodiments 323-353, wherein the liquid formulation has a nicotine concentration of about 1% (w/w) to about 25% (w/w).

Embodiment 355. The cartomizer of any one of embodiments 323-353, wherein the liquid formulation has a nicotine concentration of about 1% (w/w) to about 20% (w/w).

Embodiment 356. The cartomizer of any one of embodiments 323-353, wherein the liquid formulation has a nicotine concentration of about 1% (w/w) to about 18% (w/w).

Embodiment 357. The cartomizer of any one of embodiments 323-353, wherein the liquid formulation has a nicotine concentration of about 1% (w/w) to about 15% (w/w).

Embodiment 358. The cartomizer of any one of embodiments 323-353, wherein the liquid formulation has a nicotine concentration of about 4% (w/w) to about 12% (w/w).

Embodiment 359. The cartomizer of any one of embodiments 323-353, wherein the liquid formulation has a nicotine concentration of about 4% (w/w).

Embodiment 360. The cartomizer of any one of embodiments 323-353, wherein the liquid formulation has a nicotine concentration of about 2% (w/w).

Embodiment 361. The cartomizer of any one of embodiments 323-360, wherein the electronic cigarette is configured to generate an aerosol inhalable by a user.

Embodiment 362. The cartomizer of embodiment 361, wherein the aerosol comprises condensate of the nicotine salt.

Embodiment 363. The cartomizer of embodiment 361, wherein the aerosol comprises condensate of freebase nicotine.

Embodiment 364. The cartomizer of embodiment 361, wherein the aerosol comprises condensate of freebase nicotine and condensate of the carrier.

Embodiment 365. The cartomizer of embodiment 361, wherein the aerosol comprises condensate of freebase nicotine and condensate of the acid.

Embodiment 366. The cartomizer of any one of embodiments 361-365, wherein the aerosol comprises condensate in particle sizes from about 0.1 microns to about 5 microns.

Embodiment 367. The cartomizer of any one of embodiments 361-365, wherein the aerosol comprises condensate in particle sizes from about 0.1 microns to about 1 or 2 microns.

Embodiment 368. The cartomizer of any one of embodiments 361-365, wherein the aerosol comprises condensate in particle sizes from about 0.1 microns to about 0.7 microns.

Embodiment 369. The cartomizer of any one of embodiments 361-365, wherein the aerosol comprises condensate in particle sizes from about 0.3 microns to about 0.4 microns.

Embodiment 370. The cartomizer of any one of embodiments 361-369, wherein the nicotine salt is in an amount that forms about 0.5% to about 20% nicotine in the inhalable aerosol.

Embodiment 371. The cartomizer of any one of embodiments 361-369, wherein the nicotine salt is in an amount that forms about 1% to about 20% nicotine in the inhalable aerosol.

Embodiment 372. The cartomizer of any one of embodiments 323-371, wherein the formulation further comprises a flavorant.

Embodiment 373. The cartomizer of any one of embodiments 323-372, wherein the formulation is non-corrosive to an electronic cigarette.

Embodiment 374. The cartomizer of any one of embodiments 323-373, wherein the acid is stable at and below operating temperature or about 200° C.

Embodiment 375. The cartomizer of any one of embodiments 323-374, wherein the acid does not decompose at and below operating temperature or about 200° C.

Embodiment 376. The cartomizer of any one of embodiments 323-375, wherein the acid does not oxidize at and below operating temperature or about 200° C.

Embodiment 377. The cartomizer of any one of embodiments 323-376, wherein the formulation is non-corrosive to the electronic cigarette.

Embodiment 378. The cartomizer of any one of embodiments 323-377, wherein the formulation is non-toxic to a user of the electronic cigarette.

Embodiment 379. The cartomizer of any one of embodiments 323-378, wherein the formulation further comprises one or more additional nicotine salts in a biologically acceptable liquid carrier suitable for generating the inhalable aerosol upon heating.

Embodiment 380. The cartomizer of embodiment 379, wherein a second acid used to form the additional nicotine salt is selected from the group consisting of salicylic acid, sorbic acid, benzoic acid, pyruvic acid, lauric acid, and levulinic acid.

Embodiment 381. An electronic cigarette for generating an inhalable aerosol comprising a fluid storage compartment, a heater, and a nicotine salt liquid formulation in the fluid storage compartment, the liquid formulation comprising a nicotine salt in a biologically acceptable liquid carrier wherein an acid used to form said nicotine salt is characterized by vapor pressure >20 mmHg at 200° C., a battery, and a mouthpiece.

Embodiment 382. An electronic cigarette for generating an inhalable aerosol comprising a fluid storage compartment, a heater, and a nicotine salt liquid formulation in the fluid storage compartment, the liquid formulation comprising a nicotine salt in a biologically acceptable liquid carrier wherein an acid used to form said nicotine salt is characterized by vapor pressure of about 20 to 200 mmHg at 200° C., a battery, and a mouthpiece.

Embodiment 383. An electronic cigarette for generating an inhalable aerosol comprising a fluid storage compartment, a heater, and a nicotine salt liquid formulation in the fluid storage compartment, the liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point, a battery, and a mouthpiece.

Embodiment 384. An electronic cigarette for generating an inhalable aerosol comprising a fluid storage compartment, a heater, and a nicotine salt liquid formulation in the fluid storage compartment, the liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point, a battery, and a mouthpiece.

Embodiment 385. The electronic cigarette of any one of embodiments 381-384, wherein the heater comprises a heater chamber, a fluid wick, and a resistive heating element in contact with the fluid wick.

Embodiment 386. The electronic cigarette of any one of embodiments 381-384, wherein the mouthpiece, the heater and the fluid storage compartment form a cartomizer separable from the battery.

Embodiment 387. The electronic cigarette of any one of embodiments 381-384, wherein the heater and the fluid storage compartment form a cartomizer separable from the battery and the mouthpiece.

Embodiment 388. The electronic cigarette of any one of embodiments 381-384, wherein the fluid storage compartment is separable from the heater, the battery and the mouthpiece.

Embodiment 389. The electronic cigarette of any one of embodiments 381-383, wherein the electronic cigarette heats the nicotine salt formulation to an operating temperature from 150° C. to 250° C.

Embodiment 390. The electronic cigarette of any one of embodiments 381-383, wherein the electronic cigarette heats the nicotine salt formulation to an operating temperature from 180° C. to 220° C.

Embodiment 391. The electronic cigarette of any one of embodiments 381-383, wherein the electronic cigarette heats the nicotine salt formulation to an operating temperature of about 200° C.

Embodiment 392. The electronic cigarette of embodiment 384, wherein the operating temperature is from 150° C. to 250° C.

Embodiment 393. The electronic cigarette of embodiment 384, wherein the operating temperature is from 180° C. to 220° C.

Embodiment 394. The electronic cigarette of embodiment 384, wherein the operating temperature is about 200° C.

Embodiment 395. The electronic cigarette of any one of embodiments 381-394, wherein the aerosol comprises condensate of the nicotine salt.

Embodiment 396. The electronic cigarette of any one of embodiments 381-394, wherein the aerosol comprises condensate of freebase nicotine.

Embodiment 397. The electronic cigarette of any one of embodiments 381-394, wherein the aerosol comprises condensate of freebase nicotine and condensate of the carrier.

Embodiment 398. The electronic cigarette of any one of embodiments 381-394, wherein the aerosol comprises condensate of freebase nicotine and condensate of the acid.

Embodiment 399. The electronic cigarette of any one of embodiments 381-398, wherein the aerosol comprises condensate in particle sizes from about 0.1 microns to about 5 microns.

Embodiment 400. The electronic cigarette of any one of embodiments 381-398, wherein the aerosol comprises condensate in particle sizes from about 0.1 microns to about 1 or 2 microns.

Embodiment 401. The electronic cigarette of any one of embodiments 381-398, wherein the aerosol comprises condensate in particle sizes from about 0.1 microns to about 0.7 microns.

Embodiment 402. The electronic cigarette of any one of embodiments 381-398, wherein the aerosol comprises condensate in particle sizes from about 0.3 microns to about 0.4 microns.

Embodiment 403. The electronic cigarette of any one of embodiments 381-402, wherein the acid is a carboxylic acid.

Embodiment 404. The electronic cigarette of any one of embodiments 381-402, wherein the acid used to form said nicotine salt is an organic acid.

Embodiment 405. The electronic cigarette of embodiment 404, wherein the organic acid is monocarboxylic acid, aromatic acid, or keto acid.

Embodiment 406. The electronic cigarette of embodiment 404, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 407. The electronic cigarette of any one of embodiments 381-402, wherein the acid used to form the nicotine salt is salicylic acid.

Embodiment 408. The electronic cigarette of any one of embodiments 381-402, wherein the acid used to form the nicotine salt is benzoic acid.

Embodiment 409. The electronic cigarette of any one of embodiments 381-402, wherein the acid used to form the nicotine salt is pyruvic acid.

Embodiment 410. The electronic cigarette of any one of embodiments 381-402, wherein the acid used to form the nicotine salt is sorbic acid.

Embodiment 411. The electronic cigarette of any one of embodiments 381-402, wherein the acid used to form the nicotine salt is lauric acid.

Embodiment 412. The electronic cigarette of any one of embodiments 381-402, wherein the acid used to form the nicotine salt is levulinic acid.

Embodiment 413. The electronic cigarette of any one of embodiments 381-402, wherein said nicotine salt comprises nicotine pyruvate.

Embodiment 414. The electronic cigarette of any one of embodiments 381-402, wherein said nicotine salt comprises nicotine salicylate.

Embodiment 415. The electronic cigarette of any one of embodiments 381-402, wherein said nicotine salt comprises nicotine sorbate.

Embodiment 416. The electronic cigarette of any one of embodiments 381-402, wherein said nicotine salt comprises nicotine laurate.

Embodiment 417. The electronic cigarette of any one of embodiments 381-402, wherein said nicotine salt comprises nicotine levulinate.

Embodiment 418. The electronic cigarette of any one of embodiments 381-402, wherein said nicotine salt comprises nicotine benzoate.

Embodiment 419. The electronic cigarette of any one of embodiments 381-419, wherein the liquid carrier comprises glycerol, propylene glycol, trimethylene glycol, water, ethanol or combinations thereof.

Embodiment 420. The electronic cigarette of any one of embodiments 381-419, wherein the liquid carrier comprises propylene glycol and vegetable glycerin.

Embodiment 421. The electronic cigarette of any one of embodiments 381-419, wherein the liquid carrier comprises 20% to 50% of propylene glycol and 80% to 50% of vegetable glycerin.

Embodiment 422. The electronic cigarette of any one of embodiments 381-419, wherein the liquid carrier comprises 30% propylene glycol and 70% vegetable glycerin.

Embodiment 423. The electronic cigarette of any one of embodiments 381-422, wherein the nicotine salt is in an amount that forms about 0.5% to about 20% nicotine in the inhalable aerosol.

Embodiment 424. The electronic cigarette of any one of embodiments 381-422, wherein the nicotine salt is in an amount that forms about 1% to about 20% nicotine in the inhalable aerosol.

Embodiment 425. The electronic cigarette of any one of embodiments 381-424, wherein the liquid formulation has a nicotine concentration of about 1% (w/w) to about 25% (w/w).

Embodiment 426. The electronic cigarette of any one of embodiments 381-424, wherein the liquid formulation has a nicotine concentration of about 1% (w/w) to about 20% (w/w).

Embodiment 427. The electronic cigarette of any one of embodiments 381-424, wherein the liquid formulation has a nicotine concentration of about 1% (w/w) to about 18% (w/w).

Embodiment 428. The electronic cigarette of any one of embodiments 381-424, wherein the liquid formulation has a nicotine concentration of about 1% (w/w) to about 15% (w/w).

Embodiment 429. The electronic cigarette of any one of embodiments 381-424, wherein the liquid formulation has a nicotine concentration of about 4% (w/w) to about 12% (w/w).

Embodiment 430. The electronic cigarette of any one of embodiments 381-424, wherein the liquid formulation has a nicotine concentration of about 4% (w/w).

Embodiment 431. The electronic cigarette of any one of embodiments 381-424, wherein the liquid formulation has a nicotine concentration of about 2% (w/w).

Embodiment 432. The electronic cigarette of any one of embodiments 381-431, wherein the formulation further comprises a flavorant.

Embodiment 433. The electronic cigarette of any one of embodiments 381-432, wherein the formulation is non-corrosive to an electronic cigarette.

Embodiment 434. The electronic cigarette of any one of embodiments 381-433, wherein the acid is stable at and below operating temperature or about 200° C.

Embodiment 435. The electronic cigarette of any one of embodiments 381-434, wherein the acid does not decompose at and below operating temperature or about 200° C.

Embodiment 436. The electronic cigarette of any one of embodiments 381-435, wherein the acid does not oxidize at and below operating temperature or about 200° C.

Embodiment 437. The electronic cigarette of any one of embodiments 381-436, wherein the formulation is non-corrosive to the electronic cigarette.

Embodiment 438. The electronic cigarette of any one of embodiments 381-437, wherein the formulation is non-toxic to a user of the electronic cigarette.

Embodiment 439. The electronic cigarette of any one of embodiments 381-438, wherein the formulation further comprises one or more additional nicotine salts in a biologically acceptable liquid carrier suitable for generating the inhalable aerosol upon heating.

Embodiment 440. The electronic cigarette of embodiment 439, wherein a second acid used to form the additional nicotine salt is selected from the group consisting of salicylic acid, sorbic acid, benzoic acid, pyruvic acid, lauric acid, and levulinic acid.

Embodiment 441. A cartridge in an electronic cigarette comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure >20 mmHg at 200° C.

Embodiment 442. A cartridge in an electronic cigarette comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure of about 20 to 200 mmHg at 200° C.

Embodiment 443. A cartridge in an electronic cigarette comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point.

Embodiment 444. A cartridge in an electronic cigarette comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Embodiment 445. The cartridge of any one of embodiments 441-444, wherein the cartridge is separable from the electronic cigarette.

Embodiment 446. The cartridge of any one of embodiments 441-445, wherein the acid is a carboxylic acid.

Embodiment 447. The cartridge of any one of embodiments 441-445, wherein the acid used to form said nicotine salt is an organic acid.

Embodiment 448. The cartridge of embodiment 447, wherein the organic acid is monocarboxylic acid, aromatic acid, or keto acid.

Embodiment 449. The cartridge of embodiment 447, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 450. The cartridge of any one of embodiments 441-445, wherein the acid used to form the nicotine salt is salicylic acid.

Embodiment 451. The cartridge of any one of embodiments 441-445, wherein the acid used to form the nicotine salt is benzoic acid.

Embodiment 452. The cartridge of any one of embodiments 441-445, wherein the acid used to form the nicotine salt is pyruvic acid.

Embodiment 453. The cartridge of any one of embodiments 441-445, wherein the acid used to form the nicotine salt is sorbic acid.

Embodiment 454. The cartridge of any one of embodiments 441-445, wherein the acid used to form the nicotine salt is lauric acid.

Embodiment 455. The cartridge of any one of embodiments 441-445, wherein the acid used to form the nicotine salt is levulinic acid.

Embodiment 456. The cartridge of any one of embodiments 441-445, wherein said nicotine salt comprises nicotine pyruvate.

Embodiment 457. The cartridge of any one of embodiments 441-445, wherein said nicotine salt comprises nicotine salicylate.

Embodiment 458. The cartridge of any one of embodiments 441-445, wherein said nicotine salt comprises nicotine sorbate.

Embodiment 459. The cartridge of any one of embodiments 441-445, wherein said nicotine salt comprises nicotine laurate.

Embodiment 460. The cartridge of any one of embodiments 441-445, wherein said nicotine salt comprises nicotine levulinate.

Embodiment 461. The cartridge of any one of embodiments 441-445, wherein said nicotine salt comprises nicotine benzoate.

Embodiment 462. The cartridge of any one of embodiments 441-461, wherein the liquid carrier comprises glycerol, propylene glycol, trimethylene glycol, water, ethanol or combinations thereof.

Embodiment 463. The cartridge of any one of embodiments 441-461, wherein the liquid carrier comprises propylene glycol and vegetable glycerin.

Embodiment 464. The cartridge of any one of embodiments 441-461, wherein the liquid carrier comprises 20% to 50% of propylene glycol and 80% to 50% of vegetable glycerin.

Embodiment 465. The cartridge of any one of embodiments 441-461, wherein the liquid carrier comprises 30% propylene glycol and 70% vegetable glycerin.

Embodiment 466. The cartridge of any one of embodiments 441-465, wherein the liquid formulation has a nicotine concentration of about 1% (w/w) to about 25% (w/w).

Embodiment 467. The cartridge of any one of embodiments 441-465, wherein the liquid formulation has a nicotine concentration of about 1% (w/w) to about 20% (w/w).

Embodiment 468. The cartridge of any one of embodiments 441-465, wherein the liquid formulation has a nicotine concentration of about 1% (w/w) to about 18% (w/w).

Embodiment 469. The cartridge of any one of embodiments 441-465, wherein the liquid formulation has a nicotine concentration of about 1% (w/w) to about 15% (w/w).

Embodiment 470. The cartridge of any one of embodiments 441-465, wherein the liquid formulation has a nicotine concentration of about 4% (w/w) to about 12% (w/w).

Embodiment 471. The cartridge of any one of embodiments 441-465, wherein the liquid formulation has a nicotine concentration of about 4% (w/w).

Embodiment 472. The cartridge of any one of embodiments 441-465, wherein the liquid formulation has a nicotine concentration of about 2% (w/w).

Embodiment 473. The cartridge of any one of embodiments 441-472, wherein the formulation further comprises a flavorant.

Embodiment 474. The cartridge of any one of embodiments 441-473, wherein the formulation is non-corrosive to an electronic cigarette.

Embodiment 475. The cartridge of any one of embodiments 441-474, wherein the acid is stable at and below operating temperature or about 200° C.

Embodiment 476. The cartridge of any one of embodiments 441-475, wherein the acid does not decompose at and below operating temperature or about 200° C.

Embodiment 477. The cartridge of any one of embodiments 441-476, wherein the acid does not oxidize at and below operating temperature or about 200° C.

Embodiment 478. The cartridge of any one of embodiments 441-477, wherein the formulation is non-corrosive to the electronic cigarette.

Embodiment 479. The cartridge of any one of embodiments 441-478, wherein the formulation is non-toxic to a user of the electronic cigarette.

Embodiment 480. The cartridge of any one of embodiments 441-479, wherein the formulation further comprises one or more additional nicotine salts in a biologically acceptable liquid carrier suitable for generating the inhalable aerosol upon heating.

Embodiment 481. The cartridge of embodiment 480, wherein a second acid used to form the additional nicotine salt is selected from the group consisting of salicylic acid, sorbic acid, benzoic acid, pyruvic acid, lauric acid, and levulinic acid.

Embodiment 482. A kit comprising (a) an electronic cigarette for generating an inhalable aerosol comprising (i) a device body comprising a cartridge receptacle, (ii) a cartridge comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure >20 mmHg at 200° C., (iii) a heater, a battery, and a mouthpiece, (b) instructions for using the electronic cigarette to generate an inhalable aerosol.

Embodiment 483. A kit comprising (a) an electronic cigarette for generating an inhalable aerosol comprising (i) a device body comprising a cartridge receptacle, (ii) a cartridge comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure of about 20 to 200 mmHg at 200° C., (iii) a heater, (iv) a battery, and (v) a mouthpiece, (b) instructions for using the electronic cigarette to generate an inhalable aerosol.

Embodiment 484. A kit comprising (a) an electronic cigarette for generating an inhalable aerosol comprising (i) a device body comprising a cartridge receptacle, (ii) a cartridge comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point, (iii) a heater, (iv) a battery, and (v) a mouthpiece, and (b) instructions for using the electronic cigarette to generate an inhalable aerosol.

Embodiment 485. A kit comprising (a) an electronic cigarette for generating an inhalable aerosol comprising (i) a device body comprising a cartridge receptacle, (ii) a cartridge comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point, (iii) a heater, (iv) a battery, and (v) a mouthpiece, (b) instructions for using the electronic cigarette to generate an inhalable aerosol.

Embodiment 486. The kit of any one of embodiments 482-485, wherein the acid is a carboxylic acid.

Embodiment 487. The kit of any one of embodiments 482-485, wherein the acid used to form said nicotine salt is an organic acid.

Embodiment 488. The kit of embodiment 487, wherein the organic acid is monocarboxylic acid, aromatic acid, or keto acid.

Embodiment 489. The kit of embodiment 487, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 490. The kit of any one of embodiments 482-485, wherein the acid used to form the nicotine salt is salicylic acid.

Embodiment 491. The kit of any one of embodiments 482-485, wherein the acid used to form the nicotine salt is benzoic acid.

Embodiment 492. The kit of any one of embodiments 482-485, wherein the acid used to form the nicotine salt is pyruvic acid.

Embodiment 493. The kit of any one of embodiments 482-485, wherein the acid used to form the nicotine salt is sorbic acid.

Embodiment 494. The kit of any one of embodiments 482-485, wherein the acid used to form the nicotine salt is lauric acid.

Embodiment 495. The kit of any one of embodiments 482-485, wherein the acid used to form the nicotine salt is levulinic acid.

Embodiment 496. The kit of any one of embodiments 482-485, wherein said nicotine salt comprises nicotine pyruvate.

Embodiment 497. The kit of any one of embodiments 482-485, wherein said nicotine salt comprises nicotine salicylate.

Embodiment 498. The kit of any one of embodiments 482-485, wherein said nicotine salt comprises nicotine sorbate.

Embodiment 499. The kit of any one of embodiments 482-485, wherein said nicotine salt comprises nicotine laurate.

Embodiment 500. The kit of any one of embodiments 482-485, wherein said nicotine salt comprises nicotine levulinate.

Embodiment 501. The kit of any one of embodiments 482-485, wherein said nicotine salt comprises nicotine benzoate.

Embodiment 502. The kit of any one of embodiments 482-501, wherein the liquid carrier comprises glycerol, propylene glycol, trimethylene glycol, water, ethanol or combinations thereof.

Embodiment 503. The kit of any one of embodiments 482-501, wherein the liquid carrier comprises propylene glycol and vegetable glycerin.

Embodiment 504. The kit of any one of embodiments 482-501, wherein the liquid carrier comprises 20% to 50% of propylene glycol and 80% to 50% of vegetable glycerin.

Embodiment 505. The kit of any one of embodiments 482-501, wherein the liquid carrier comprises 30% propylene glycol and 70% vegetable glycerin.

Embodiment 506. The kit of any one of embodiments 482-505, wherein the liquid formulation has a nicotine concentration of about 1% (w/w) to about 25% (w/w).

Embodiment 507. The kit of any one of embodiments 482-505, wherein the liquid formulation has a nicotine concentration of about 1% (w/w) to about 20% (w/w).

Embodiment 508. The kit of any one of embodiments 482-505, wherein the liquid formulation has a nicotine concentration of about 1% (w/w) to about 18% (w/w).

Embodiment 509. The kit of any one of embodiments 482-505, wherein the liquid formulation has a nicotine concentration of about 1% (w/w) to about 15% (w/w).

Embodiment 510. The kit of any one of embodiments 482-505, wherein the liquid formulation has a nicotine concentration of about 4% (w/w) to about 12% (w/w).

Embodiment 511. The kit of any one of embodiments 482-505, wherein the liquid formulation has a nicotine concentration of about 4% (w/w).

Embodiment 512. The kit of any one of embodiments 482-505, wherein the liquid formulation has a nicotine concentration of about 2% (w/w).

Embodiment 513. The kit of any one of embodiments 482-512, wherein the formulation further comprises a flavorant.

Embodiment 514. The kit of any one of embodiments 482-513, wherein the formulation is non-corrosive to an electronic cigarette.

Embodiment 515. The kit of any one of embodiments 482-514, wherein the acid is stable at and below operating temperature or about 200° C.

Embodiment 516. The kit of any one of embodiments 482-515, wherein the acid does not decompose at and below operating temperature or about 200° C.

Embodiment 517. The kit of any one of embodiments 482-516, wherein the acid does not oxidize at and below operating temperature or about 200° C.

Embodiment 518. The kit of any one of embodiments 482-517, wherein the formulation is non-corrosive to the electronic cigarette.

Embodiment 519. The kit of any one of embodiments 482-518, wherein the formulation is non-toxic to a user of the electronic cigarette.

Embodiment 520. The kit of any one of embodiments 482-519, wherein the formulation further comprises one or more additional nicotine salts in a biologically acceptable liquid carrier suitable for generating the inhalable aerosol upon heating.

Embodiment 521. The kit of embodiment 520, wherein a second acid used to form the additional nicotine salt is selected from the group consisting of salicylic acid, sorbic acid, benzoic acid, pyruvic acid, lauric acid, and levulinic acid.

Embodiment 522. A cartridge comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure >20 mmHg at 200° C.

Embodiment 523. A cartridge comprising a fluid storage compartment, wherein the fluid storage compartment stores a nicotine salt liquid formulation comprising a nicotine salt in a biologically acceptable liquid carrier wherein an acid used to form said nicotine salt is characterized by vapor pressure of about 20 to 200 mmHg at 200° C.

Embodiment 524. A cartridge comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point <160° C., a boiling point >160° C., and at least a 50-degree difference between the melting point and the boiling point.

Embodiment 525. A cartridge comprising a fluid storage compartment, wherein the fluid storage compartment stores a nicotine salt liquid formulation comprising a nicotine salt in a biologically acceptable liquid carrier wherein an acid used to form said nicotine salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

Embodiment 526. The cartridge of any one of embodiments 523-526, wherein the cartridge can be connected to an electronic cigarette.

Embodiment 527. The cartridge of any one of embodiments 523-527, wherein the acid is a carboxylic acid.

Embodiment 528. The cartridge of any one of embodiments 523-527, wherein the acid used to form said nicotine salt is an organic acid.

Embodiment 529. The cartridge of embodiment 529, wherein the organic acid is monocarboxylic acid, aromatic acid, or keto acid.

Embodiment 530. The cartridge of embodiment 529, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, or malic acid.

Embodiment 531. The cartridge of any one of embodiments 523-527, wherein the acid used to form the nicotine salt is salicylic acid.

Embodiment 532. The cartridge of any one of embodiments 523-527, wherein the acid used to form the nicotine salt is benzoic acid.

Embodiment 533. The cartridge of any one of embodiments 523-527, wherein the acid used to form the nicotine salt is pyruvic acid.

Embodiment 534. The cartridge of any one of embodiments 523-527, wherein the acid used to form the nicotine salt is sorbic acid.

Embodiment 535. The cartridge of any one of embodiments 523-527, wherein the acid used to form the nicotine salt is lauric acid.

Embodiment 536. The cartridge of any one of embodiments 523-527, wherein the acid used to form the nicotine salt is levulinic acid.

Embodiment 537. The cartridge of any one of embodiments 523-527, wherein said nicotine salt comprises nicotine pyruvate.

Embodiment 538. The cartridge of any one of embodiments 523-527, wherein said nicotine salt comprises nicotine salicylate.

Embodiment 539. The cartridge of any one of embodiments 523-527, wherein said nicotine salt comprises nicotine sorbate.

Embodiment 540. The cartridge of any one of embodiments 523-527, wherein said nicotine salt comprises nicotine laurate.

Embodiment 541. The cartridge of any one of embodiments 523-527, wherein said nicotine salt comprises nicotine levulinate.

Embodiment 542. The cartridge of any one of embodiments 523-527, wherein said nicotine salt comprises nicotine benzoate.

Embodiment 543. The cartridge of any one of embodiments 523-543, wherein the liquid carrier comprises glycerol, propylene glycol, trimethylene glycol, water, ethanol or combinations thereof.

Embodiment 544. The cartridge of any one of embodiments 523-543, wherein the liquid carrier comprises propylene glycol and vegetable glycerin.

Embodiment 545. The cartridge of any one of embodiments 523-543, wherein the liquid carrier comprises 20% to 50% of propylene glycol and 80% to 50% of vegetable glycerin.

Embodiment 546. The cartridge of any one of embodiments 523-543, wherein the liquid carrier comprises 30% propylene glycol and 70% vegetable glycerin.

Embodiment 547. The cartridge of any one of embodiments 523-547, wherein the liquid formulation has a nicotine concentration of about 1% (w/w) to about 25% (w/w).

Embodiment 548. The cartridge of any one of embodiments 523-547, wherein the liquid formulation has a nicotine concentration of about 1% (w/w) to about 20% (w/w).

Embodiment 549. The cartridge of any one of embodiments 523-547, wherein the liquid formulation has a nicotine concentration of about 1% (w/w) to about 18% (w/w).

Embodiment 550. The cartridge of any one of embodiments 523-547, wherein the liquid formulation has a nicotine concentration of about 1% (w/w) to about 15% (w/w).

Embodiment 551. The cartridge of any one of embodiments 523-547, wherein the liquid formulation has a nicotine concentration of about 4% (w/w) to about 12% (w/w).

Embodiment 552. The cartridge of any one of embodiments 523-547, wherein the liquid formulation has a nicotine concentration of about 4% (w/w).

Embodiment 553. The cartridge of any one of embodiments 523-547, wherein the liquid formulation has a nicotine concentration of about 2% (w/w).

Embodiment 554. The cartridge of any one of embodiments 523-553, wherein the formulation further comprises a flavorant.

Embodiment 555. The cartridge of any one of embodiments 523-554, wherein the formulation is non-corrosive to an electronic cigarette.

Embodiment 556. The cartridge of any one of embodiments 523-555, wherein the acid is stable at and below operating temperature or about 200° C.

Embodiment 557. The cartridge of any one of embodiments 523-556, wherein the acid does not decompose at and below operating temperature or about 200° C.

Embodiment 558. The cartridge of any one of embodiments 523-557, wherein the acid does not oxidize at and below operating temperature or about 200° C.

Embodiment 559. The cartridge of any one of embodiments 523-558, wherein the formulation is non-corrosive to the electronic cigarette.

Embodiment 560. The cartridge of any one of embodiments 523-559, wherein the formulation is non-toxic to a user of the electronic cigarette.

Embodiment 561. The cartridge of any one of embodiments 523-560, wherein the formulation further comprises one or more additional nicotine salts in a biologically acceptable liquid carrier suitable for generating the inhalable aerosol upon heating.

Embodiment 562. The cartridge of embodiment 561, wherein a second acid used to form the additional nicotine salt is selected from the group consisting of salicylic acid, sorbic acid, benzoic acid, pyruvic acid, lauric acid, and levulinic acid.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following embodiments define the scope of the invention and that methods and structures within the scope of these embodiments and their equivalents be covered thereby.

What is claimed is:

1. An electronic cigarette cartridge comprising a nicotine salt liquid formulation, wherein:
(a) the nicotine salt liquid formulation comprises a salt of nicotine and an organic acid in a liquid carrier;
(b) the salt is present in an amount that forms a nicotine concentration of 0.5% (w/w) to 20% (w/w) in the nicotine salt liquid formulation; and
(c) the nicotine salt liquid formulation generates an inhalable aerosol upon heating in the electronic cigarette.

2. The electronic cigarette cartridge of claim 1, wherein the liquid carrier comprises glycerol, propylene glycol, trimethylene glycol, water, ethanol or combinations thereof.

3. The electronic cigarette cartridge of claim 2, wherein the liquid carrier comprises propylene glycol and glycerol.

4. The electronic cigarette cartridge of claim 1, wherein the salt is present in an amount that forms a nicotine concentration of 1% (w/w) to 18% (w/w) in the nicotine salt liquid formulation.

5. The electronic cigarette cartridge of claim 4, wherein the salt is present in an amount that forms a nicotine concentration of 3% (w/w) to 15% (w/w) in the nicotine salt liquid formulation.

6. The electronic cigarette cartridge of claim 4, wherein the salt is present in an amount that forms a nicotine concentration of 4% (w/w) to 12% (w/w) in the nicotine salt liquid formulation.

7. The electronic cigarette cartridge of claim 1, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, malic acid, or a combination of two or more thereof.

8. The electronic cigarette cartridge of claim 1, wherein the nicotine salt liquid formulation further comprises a flavorant.

9. The electronic cigarette cartridge of claim 1, wherein the nicotine salt liquid formulation further comprises one or more additional organic acids.

10. An electronic cigarette comprising the electronic cigarette cartridge of claim 1 and a heater.

11. An electronic cigarette comprising a nicotine salt liquid formulation and a heater, wherein the nicotine salt liquid formulation comprises about 0.5% (w/w) to 20% (w/w) of a nicotine salt, wherein the nicotine salt comprises nicotine and an organic acid.

12. The electronic cigarette of claim 11, wherein the organic acid is formic acid, acetic acid, propionic acid, butyric acid, valeric acid, caproic acid, caprylic acid, capric acid, citric acid, lauric acid, myristic acid, palmitic acid, stearic acid, oleic acid, linoleic acid, linolenic acid, phenylacetic acid, benzoic acid, pyruvic acid, levulinic acid, tartaric acid, lactic acid, malonic acid, succinic acid, fumaric acid, finnaric acid, gluconic acid, saccharic acid, salicylic acid, sorbic acid, malonic acid, malic acid, or a combination of two or more thereof.

13. A method of providing nicotine to a user, the method comprising heating a nicotine salt liquid formulation in an electronic cigarette to produce an inhalable aerosol, and inhalation of the aerosol by the user; wherein:
(i) the nicotine salt liquid formulation comprises a nicotine salt in a liquid carrier, wherein the nicotine salt comprises nicotine and an organic acid; and
(ii) the nicotine salt is present in an amount that forms a nicotine concentration of 0.5% (w/w) to 20% (w/w) in the nicotine salt liquid formulation.

14. The method of claim 13, wherein the liquid carrier comprises glycerol, propylene glycol, trimethylene glycol, water, ethanol or combinations thereof.

15. The method of claim 14, wherein the liquid carrier comprises propylene glycol and glycerol.

16. The method of claim 13, wherein the salt is present in an amount that forms a nicotine concentration of 1% (w/w) to 18% (w/w) in the nicotine salt liquid formulation.

17. The method of claim 16, wherein the salt is present in an amount that forms a nicotine concentration of 3% (w/w) to 15% (w/w) in the nicotine salt liquid formulation.

18. The method of claim 16, wherein the salt is present in an amount that forms a nicotine concentration of 4% (w/w) to 12% (w/w) in the nicotine salt liquid formulation.

19. The method of claim 13, wherein the nicotine salt liquid formulation further comprises a flavorant.

20. The method of claim 13, wherein the nicotine salt liquid formulation further comprises one or more additional organic acids.
